(19) United States
(12) Patent Application Publication
(10) Pub. No.: US 2024/0013392 A1
(43) Pub. Date: Jan. 11, 2024

(54) PROCESSOR DEVICE, MEDICAL IMAGE PROCESSING DEVICE, MEDICAL IMAGE PROCESSING SYSTEM, AND ENDOSCOPE SYSTEM

(71) Applicant: FUJIFILM Corporation, Tokyo (JP)

(72) Inventor: Takaaki SHIMIZU, Kanagawa (JP)

(73) Assignee: FUJIFILM Corporation, Tokyo (JP)

(21) Appl. No.: 18/474,251

(22) Filed: Sep. 26, 2023

Related U.S. Application Data
(63) Continuation of application No. PCT/JP2022/014916, filed on Mar. 28, 2022.

(30) Foreign Application Priority Data
Mar. 31, 2021 (JP) 2021-061946

(51) Int. Cl. G06T 7/00; G06T 5/50; G06V 10/143; G16H 30/20; G16H 30/40; G06F 3/14

(52) U.S. Cl. CPC G06T 7/0012; G06T 5/50; G06V 10/143; G16H 30/20; G16H 30/40; G06F 3/14; G06T 2207/20221

(57) ABSTRACT

The medical image processing system includes the processor device and the medical image processing device. The processor device generates an identification information-assigned medical image generated by assigning a part of data constituting a medical image as identification information indicating a type of the medical image, and the medical image processing device acquires the identification information-assigned medical image, identifies the type of the medical image, and performs image processing corresponding to the type of the medical image. The endoscope system includes a light source, an endoscope, and the medical image processing system.

### CROSS-REFERENCE TO RELATED APPLICATIONS

This application is a Continuation of PCT International Application No. PCT/JP2022/014916 filed on 28 Mar. 2022, which claims priority under 35 U.S.C § 119(a) to Japanese Patent Application No. 2021-061946 filed on 31 Mar. 2021. The above application is hereby expressly incorporated by reference, in its entirety, into the present application.

### BACKGROUND OF THE INVENTION

### 1. Field of the Invention

The present invention relates to a processor device, a medical image processing device, a medical image processing system, and an endoscope system.

### 2. Description of the Related Art

In the medical field, diagnosis using an endoscope system that comprises a light source device, an endoscope, and a processor device has been widely performed. In the diagnosis using the endoscope system, there is a case where various types of diagnosis support information related to a surface structure, a mucous membrane surface layer, or the like of an observation target may be obtained using an image (hereinafter, referred to as an endoscope image) obtained by imaging the observation target with the endoscope through image-enhanced observation (image enhanced endoscopy (IEE)) in which illumination light or the like is devised.

In the diagnosis using the IEE, there is a case where an appropriate diagnosis may be made by acquiring a plurality of types of endoscope images obtained using a plurality of types of illumination light or the like, and by comparing in detail or superimposing these endoscope images. For example, an endoscope system is known that can prevent overlooking of a lesion or the like and can accurately determine the severity or progression of a disease in an endoscopic examination by acquiring a normal image signal using illumination light of white light and a special image signal using illumination light of special light having a spectrum different from that of white light (JP2020-065685A).

### SUMMARY OF THE INVENTION

Appropriate diagnosis support information can be obtained by performing different image processing between the endoscope image based on the normal image signal obtained using white light and the endoscope image based on the special image signal obtained using special light. That is, it is preferable to, in a case where the type of the endoscope image is set to correspond to the type of illumination light when the endoscope image is acquired, perform image processing on the endoscope image selected according to the type of the endoscope image.

The type of illumination light is decided on based on a signal transmitted from the processor device to the light source device, and the processor device has information on both the endoscope image and the type thereof. Therefore, in a case where the endoscope image is transmitted from the processor device to an external device and the external device performs image processing, the type of the endoscope image also needs to be transmitted from the processor device to the external device at the same time. At this time, in order to prevent the correspondence between the endoscope image and the type of the endoscope image from being lost, in a case where the type of the endoscope image is recorded in a header portion or the like of an information container that stores the endoscope image and is transmitted, the endoscope image cannot be transmitted as a general video signal, such as a digital visual interface (DVI), and cannot be received by a general-purpose personal computer (hereinafter, referred to as a PC) in many cases. In addition, in many cases, it is also difficult for the PC to perform transmission through another signal line in order to prevent the correspondence from being lost.

Meanwhile, since image processing by the PC is widely performed in various ways, there has been a demand for more simply discriminating and handling the endoscope image through the PC.

An object of the present invention is to provide a processor device, a medical image processing device, a medical image processing system, and an endoscope system capable of easily discriminating the type of an endoscope image.

According to the present invention, there is provided a processor device comprising: a first processor, in which the first processor is configured to: acquire a plurality of types of medical images with different imaging conditions; and generate an identification information-assigned medical image in which a part of data constituting the medical image is assigned as identification information indicating a type of the medical image by changing the part of the data constituting the medical image, or by changing the part of the data constituting the medical image in at least one type of the medical image and not changing the part of the data constituting the medical image in another type of the medical image, according to the type of the medical image.

It is preferable that the data constituting the medical image is data constituting a preset region of the medical image.

It is preferable that the data constituting the medical image is a pixel value.

It is preferable that the plurality of types of medical images include a display image for display on a display and an analysis image for analysis related to diagnostic information.

It is preferable that the first processor is configured to assign the identification information to the analysis image by changing a part of data constituting the analysis image, and to assign the identification information to the display image without changing a portion of data constituting the display image, the portion corresponding to the data assigned as the identification information in the analysis image.

It is preferable that the first processor is configured to assign the identification information to the display image by changing a part of data constituting the display image, and to assign the identification information to the analysis image without changing a portion of data constituting the analysis image, the portion corresponding to the data assigned as the identification information in the display image.

It is preferable that the imaging condition is a spectrum of illumination light.

In addition, according to the present invention, there is provided a medical image processing device comprising: a second processor, in which the second processor is configured to: acquire a plurality of types of identification information-assigned medical images in which a part of data constituting a medical image is assigned as identification information; recognize the type of the identification information-assigned medical image based on the identification information; and perform control to display the identification information-assigned medical image on a display based on the type of the identification information-assigned medical image.

It is preferable that the plurality of types of identification information-assigned medical images include a display image for display on the display and an analysis image for analysis related to diagnostic information.

It is preferable that the second processor is configured to display the display image on a main screen of the display, and to decide whether or not to display the analysis image on a sub screen of the display based on the type of the identification information-assigned medical image and display, on the sub screen of the display, the identification information-assigned medical image that is decided to be displayed.

It is preferable that the second processor is configured to perform image processing set for each type of the identification information-assigned medical image on the identification information-assigned medical image based on the type of the identification information-assigned medical image.

It is preferable that the second processor is configured to, in a case where the identification information-assigned medical image is the display image, perform display image processing on the display image, and to, in a case where the identification information-assigned medical image is the analysis image, perform analysis image processing on the analysis image.

It is preferable that the second processor is configured to perform the analysis image processing using a machine learning-based analysis model.

It is preferable that the second processor is configured to create an analysis result image indicating a result of the analysis image processing, and to generate a superimposition image by superimposing the analysis result image on the display image.

Further, according to the present invention, there is provided a medical image processing system comprising: the processor device; and the medical image processing device, in which the second processor is configured to acquire the plurality of types of identification information-assigned medical images generated by the first processor.

Further, according to the present invention, there is provided a medical image processing system comprising: the processor device; and the medical image processing device, in which the processor device is configured to acquire the analysis result image indicating the result of the analysis image processing and created by the second processor.

It is preferable that the processor device is configured to superimpose the analysis result image on the display image.

It is preferable that the processor device is configured to adjust a frame rate of the identification information-assigned medical image, and the medical image processing device is configured to acquire the identification information-assigned medical image of which the frame rate is adjusted.

It is preferable that the processor device or the medical image processing device is configured to adjust a frame rate of an image for display on a display.

Further, according to the present invention, there is provided an endoscope system comprising: a plurality of light sources that emit light rays having wavelength ranges different from each other; an endoscope that images a subject illuminated with illumination light emitted from the plurality of light sources; and the medical image processing system, in which the processor device includes a light source processor that is configured to perform control to emit each of a plurality of types of the illumination light having different combinations of light intensity ratios between the plurality of light sources from each other.

According to the present invention, it is possible to easily discriminate the type of the endoscope image.

### BRIEF DESCRIPTION OF THE DRAWINGS

FIG. 1 is an external view of an endoscope system.

FIG. 2 is a block diagram showing a function of the endoscope system.

FIG. 3 is an explanatory diagram illustrating four colors of LEDs provided in a light source unit.

FIG. 4 is a graph showing spectra of violet light V, blue light B, green light G, and red light R.

FIG. 5 is a graph showing a spectrum of a first illumination light.

FIG. 6 is an explanatory diagram illustrating a type and an order of an endoscope image captured by the endoscope system.

FIG. 7 is an image diagram showing an identification information-assigned endoscope image provided with identification information.

FIG. 8 is an image diagram showing an endoscope image including an observation target portion and a mask portion.

FIG. 9A is an image diagram showing a first identification information-assigned endoscope image provided with first identification information, and FIG. 9B is an image diagram showing a second identification information-assigned endoscope image provided with second identification information.

FIG. 10 is an explanatory diagram illustrating the type of the endoscope image captured by the endoscope system and the identification information.

FIG. 11 is an explanatory diagram illustrating the type and an imaging order of the endoscope image captured by the endoscope system and the identification information.

FIG. 12 is an explanatory diagram illustrating a case where the identification information is assigned to an analysis image.

FIG. 13 is an explanatory diagram illustrating a case where the identification information is assigned to a display image.

FIG. 14 is a block diagram showing a function of the medical image processing device.

FIG. 15 is an explanatory diagram illustrating various images and a flow of processing in the medical image processing device.

FIG. 16 is an image diagram in a case where the analysis image is displayed on a sub screen of a display.

FIG. 17 is an image diagram in a case where an image is not displayed on the sub screen of the display.

FIG. 18 is an image diagram in a case where a past image is displayed on the sub screen of the display.

FIG. 19 is an explanatory diagram illustrating a function of a frame rate conversion section that duplicates the display image and the analysis image to adjust a frame rate.

FIG. 20 is an explanatory diagram illustrating a function of the frame rate conversion section that duplicates the display image to adjust the frame rate.

FIG. 21 is an explanatory diagram illustrating a third identification information-assigned endoscope image generated by assigning the identification information based on the type of the endoscope image to a complementary frame image.

FIG. 22 is an explanatory diagram illustrating the third identification information-assigned endoscope image generated by assigning the identification information based on the type of the endoscope image and information on an original image to the complementary frame image.

FIG. 23 is an explanatory diagram illustrating the third identification information-assigned endoscope image generated by assigning the identification information based on the type and the imaging order of the endoscope image to the display image, the analysis image, and the complementary frame image.

FIG. 24 is a flowchart showing a series of flows of endoscope image processing in a medical image processing system and the endoscope system.

FIG. 25 is an explanatory diagram illustrating a case where the medical image processing device is provided in a diagnosis support apparatus.

FIG. 26 is an explanatory diagram illustrating a case where the medical image processing device is provided in a medical service support apparatus.

### DESCRIPTION OF THE PREFERRED EMBODIMENTS

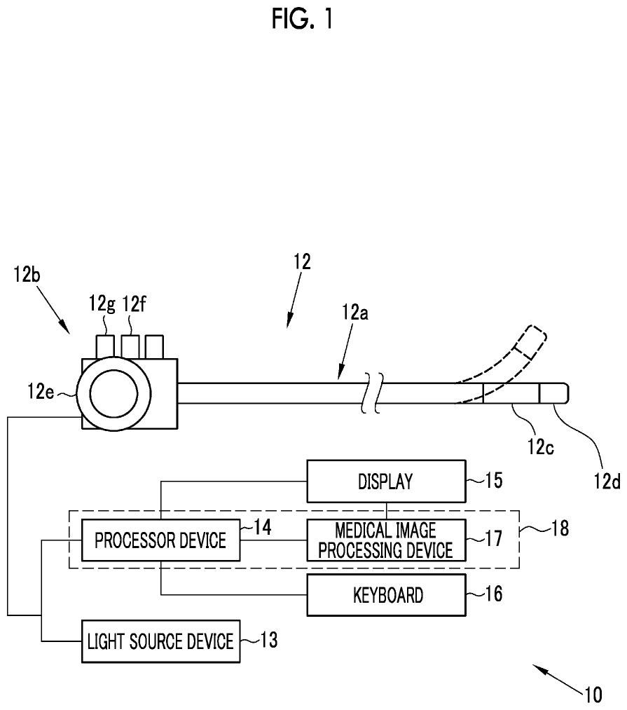

As shown in FIG. 1, an endoscope system 10 includes an endoscope 12, a light source device 13, a processor device 14, a display 15, a keyboard 16, and a medical image processing device 17. The endoscope 12 is optically connected to the light source device 13 and is electrically connected to the processor device 14. The processor device 14 is connected to the medical image processing device 17. The medical image processing device 17 receives, from the processor device 14, an endoscope image to which identification information is assigned, and performs various types of image processing including image analysis by machine learning or the like. In the present embodiment, a medical image is an endoscope image.

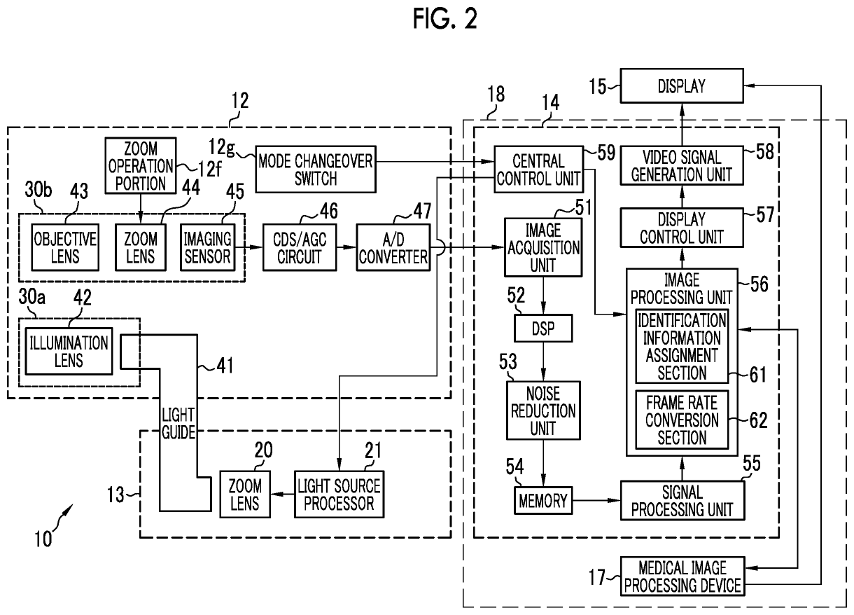

The endoscope 12 includes an insertion part 12a to be inserted into a body of a subject to be examined having an observation target, an operation part 12b provided at a proximal end part of the insertion part 12a, and a bendable portion 12c and a distal end portion 12d provided on a distal end side of the insertion part 12a. By operating an angle knob 12e (see FIG. 2) of the operation part 12b, the bendable portion 12c performs a bending movement. The distal end portion 12d is directed in a desired direction by the bending movement of the bendable portion 12c.

The operation part 12b includes, in addition to the angle knob 12e, a zoom operation portion 12f for changing an imaging magnification and a mode changeover switch 12g used for a switching operation of an observation mode. The switching operation of the observation mode or a zoom operation may be an operation or an instruction using the keyboard 16, a footswitch (not shown), or the like in addition to the mode changeover switch 12g or the zoom operation portion 12f.

The endoscope system 10 has three observation modes: a normal observation mode, a special observation mode, and a diagnosis support observation mode. The normal observation mode is a mode in which a normal image, which is a natural color-tone image obtained by capturing an image of the observation target using white light as illumination light, is displayed on the display 15. The special observation mode includes a first special observation mode. The first special observation mode is a mode in which a first image in which surface layer information, such as surface layer blood vessels, is enhanced is displayed on the display 15.

The diagnosis support observation mode is a mode in which a superimposition image obtained by superimposing an analysis result image, which shows a result of image analysis through display, on the normal image is displayed on the display 15. The result of the image analysis is diagnosis support information for supporting diagnosis of a doctor or the like and is obtained by the image analysis using the first image. Therefore, the analysis result image includes diagnosis support information related to a lesion or the like obtained by the image analysis using the first image. In the diagnosis support observation mode, in a case where a lesion or the like is detected by the image analysis using the first image, the superimposition image in which the analysis result image indicating the diagnosis support information, such as the position of the lesion, is superimposed on the normal image is displayed on the display 15. At the time of activation of the endoscope system 10, the diagnosis support observation mode is selected.

The processor device 14 is electrically connected to the display 15 and the keyboard 16. The display 15 displays, for example, the normal image, the first image, the superimposition image, and/or information accompanying these images. The keyboard 16 functions as a user interface that receives an input operation, such as function settings. An external recording unit (not shown) that records images, image information, or the like may be connected to the processor device 14.

As shown in FIG. 2, the light source device 13 emits illumination light with which the observation target is irradiated, and comprises a light source unit 20 and a light source processor 21 that controls the light source unit 20. The light source unit 20 includes, for example, a semiconductor light source, such as a plurality of colors of light emitting diodes (LEDs), a combination of a laser diode and a phosphor, or a xenon lamp or a halogen light source. In addition, the light source unit 20 includes, for example, an optical filter for adjusting the wavelength range of light emitted by the LED or the like. The light source processor 21 turns on/off each LED or the like or adjusts the drive current and the drive voltage of each LED or the like, thereby controlling the amount of illumination light. Further, the light source processor 21 changes the optical filter or the like, thereby controlling the wavelength range of illumination light.

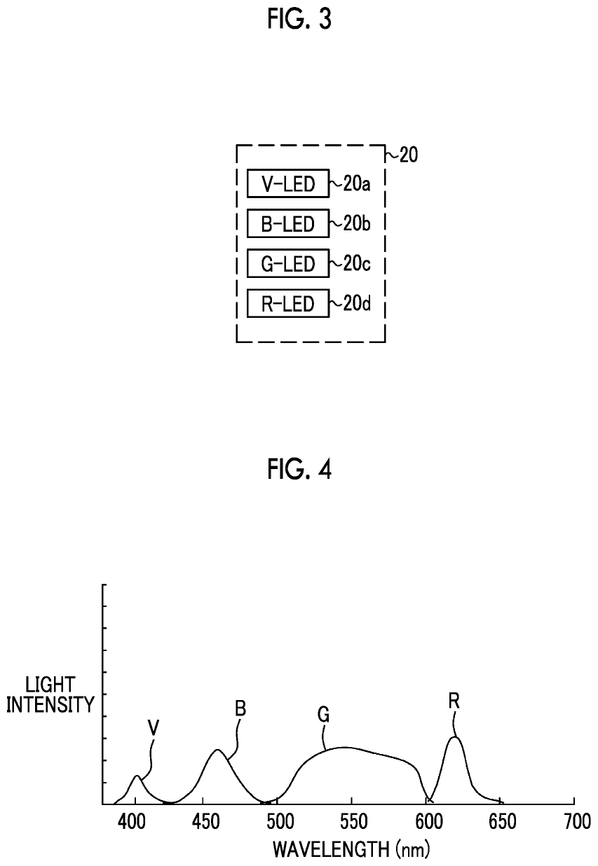

As shown in FIG. 3, in the present embodiment, the light source unit 20 has four colors of LEDs, that is, a violet light emitting diode (V-LED) 20a, a blue light emitting diode (B-LED) 20b, a green light emitting diode (G-LED) 20c, and a red light emitting diode (R-LED) 20d.

As shown in FIG. 4, the V-LED 20a generates violet light V having a central wavelength of 410±10 nm and a wavelength range of 380 to 420 nm. The B-LED 20b generates blue light B having a central wavelength of 450±10 nm and a wavelength range of 420 to 500 nm. The G-LED 20c generates green light G having a wavelength range of 480 to 600 nm. The R-LED 20d generates red light R having a central wavelength of 620 to 630 nm and a wavelength range of 600 to 650 nm.

The light source processor 21 controls the V-LED 20a, the B-LED 20b, the G-LED 20c, and the R-LED 20d. The light source processor 21 controls the respective LEDs 20a to 20d to emit normal light of which the combination of light intensity ratios between violet light V, blue light B, green light G, and red light R is Vc:Bc:Gc:Rc in the normal observation mode.

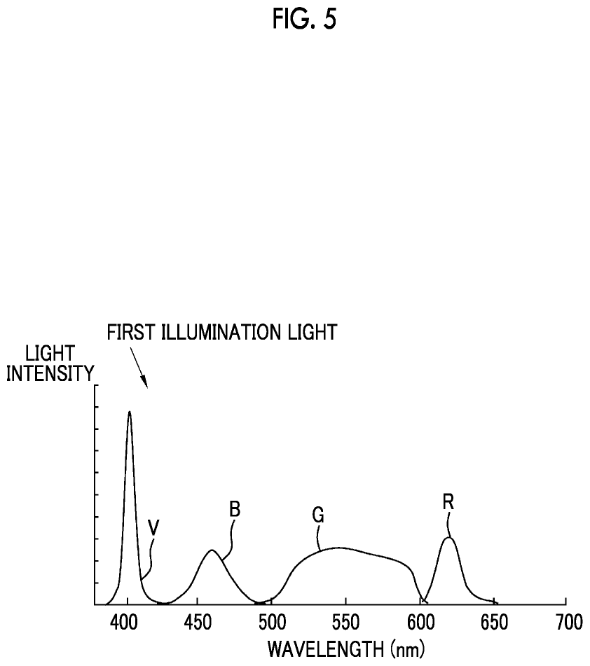

The light source processor 21 controls the respective LEDs 20a to 20d to emit first illumination light of which the combination of the light intensity ratios between violet light V, blue light B, green light G, and red light R is Vs1:Bs1:Gs1:Rs1 in the special observation mode. It is preferable that the first illumination light enhances surface layer blood vessels. For this purpose, it is preferable that the light intensity of violet light V of the first illumination light is set to be higher than the light intensity of blue light B. For example, as shown in FIG. 5, a ratio of a light intensity Vs1 of violet light V to a light intensity Bs1 of blue light B is set to “4:1”.

In the present specification, the combinations of the light intensity ratios include a case where the ratio of at least one semiconductor light source is zero (0). Therefore, the combinations of the light intensity ratios include a case where any one or two or more of the semiconductor light sources are not turned on. For example, a case where only one semiconductor light source is turned on and the other three semiconductor light sources are not turned on as in a case where the combination of the light intensity ratios between violet light V, blue light B, green light G, and red light R is 1:0:0:0 is also regarded as having light intensity ratios and is one of the combinations of the light intensity ratios.

As described above, the combinations of the light intensity ratios between violet light V, blue light B, green light G, and red light R, which are emitted in the normal observation mode or the special observation mode, that is, the types of illumination light, are different from each other. In the diagnosis support observation mode, different types of Illumination light are automatically switched and emitted. An observation mode using a different type of illumination light of which the combination of the light intensity ratios is different from those of the illumination light used in these observation modes may be used.

In a case where the diagnosis support observation mode is set, the light source processor 21 switches and emits a specific type of illumination light. Specifically, a normal light period in which the normal light is continuously emitted and a first illumination light period in which the first illumination light is continuously emitted are alternately repeated. Specifically, the normal light period in which the normal light is emitted is performed for a predetermined number of frames, and then the first illumination light period in which the first illumination light is emitted is performed for a predetermined number of frames. Thereafter, the normal light period starts again, and a set of the normal light period and the first illumination light period is repeated.

“Frame” refers to a unit for controlling an imaging sensor 45 (see FIG. 2) that captures an image of the observation target, and for example, “one frame” refers to a period of time including at least an exposure period for exposing the imaging sensor 45 to light from the observation target and a readout period for reading out image signals. In the present embodiment, various periods, such as the normal light period or the first illumination light period, are each determined in correspondence with the “frame” which is a unit of imaging.

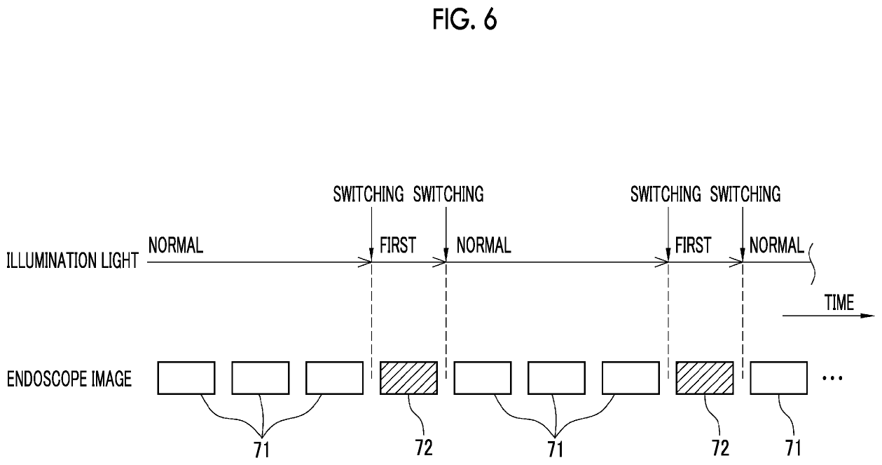

As shown in FIG. 6, in the diagnosis support observation mode, the normal light period in which the normal light is emitted is performed for three frames, and then the first illumination light period in which the first illumination light is emitted is performed for one frame. Thereafter, the normal light period starts again, and a set of the normal light period and the first illumination light period is repeated for four frames. Therefore, after three normal images 71 are consecutively captured during three frames of the normal light period, one first image 72 is captured during the first illumination light period. In FIG. 6, the first image 72 is shaded. Thereafter, the period returns to the normal light period, and this pattern is continuously repeated.

The light emitted from each of the LEDs 20a to 20d is incident on a light guide 41 via an optical path coupling portion (not shown) that includes a mirror, a lens, or the like. The light guide 41 is incorporated into the endoscope 12 and a universal cord (a cord that connects the endoscope 12 to the light source device 13 and the processor device 14). The light guide 41 propagates the light from the optical path coupling portion to the distal end portion 12d of the endoscope 12.

An illumination optical system 30a and an imaging optical system 30b are provided in the distal end portion 12d of the endoscope 12. The illumination optical system 30a includes an illumination lens 42, and the observation target is irradiated with illumination light propagated by the light guide 41 via the illumination lens 42. The imaging optical system 30b includes an objective lens 43, a zoom lens 44, and the imaging sensor 45. Various types of light, such as reflected light, scattered light, and fluorescence, from the observation target are incident on the imaging sensor 45 via the objective lens 43 and the zoom lens 44. As a result, an image of the observation target is formed on the imaging sensor 45. The zoom lens 44 is freely moved between a telephoto end and a wide end by operating the zoom operation portion 12f, and magnifies and reduces the observation target of which the image is formed on the imaging sensor 45.

The imaging sensor 45 is a color imaging sensor provided with any of a red (R) color filter, a green (G) color filter, or a blue (B) color filter for each pixel, and captures the image of the observation target and outputs image signals of respective RGB colors. A charge coupled device (CCD) imaging sensor or a complementary metal-oxide semiconductor (CMOS) imaging sensor can be used as the imaging sensor 45. Alternatively, a complementary color imaging sensor provided with complementary color filters, that is, cyan (C), magenta (M), yellow (Y), and green (G), may be used instead of the imaging sensor 45 provided with primary color filters. In a case where the complementary color imaging sensor is used, the image signals of four colors, that is, CMYG, are output. Therefore, the same RGB image signals as those of the imaging sensor 45 can be obtained by converting the image signals of the four colors, that is, CMYG, into the image signals of the three colors, that is, RGB, through the complementary color-primary color conversion. Alternatively, a monochrome imaging sensor that is not provided with color filters may be used instead of the imaging sensor 45.

The imaging sensor 45 is driven and controlled by an imaging control unit (not shown). The central control unit 59 (see FIG. 3) controls the light emission of the light source unit 20 through the light source processor 21 in synchronization with the imaging control unit to perform control so as to capture the image of the observation target illuminated with the normal light in the normal observation mode. As a result, a Bc image signal is output from the B pixel of the imaging sensor 45, a Gc image signal is output from the G pixel, and an Rc image signal is output from the R pixel. In the special observation mode or the diagnosis support observation mode, the central control unit 59 (see FIG. 3) controls the light emission of the light source unit 20 to control the imaging sensor 45 so as to capture the image of the observation target illuminated with the special light. As a result, in the first special observation mode, a Bs1 image signal is output from the B pixel of the imaging sensor 45, a Gs1 image signal is output from the G pixel, and an Rs1 image signal is output from the R pixel.

A correlated double sampling/automatic gain control (CDS/AGC) circuit 46 performs correlated double sampling (CDS) or automatic gain control (AGC) on analog image signals obtained from the imaging sensor 45. The image signals that have passed through the CDS/AGC circuit 46 are converted into digital image signals by an analog/digital (A/D) converter 47. The digital image signals after the A/D conversion are input to the processor device 14.

In the processor device 14, a program related to processing, such as image processing, is stored in a program memory (not shown). In the processor device 14, the program stored in the program memory is operated by the central control unit 59 composed of an image processor, which is a first processor, or the like, whereby functions of an image acquisition unit 51, a digital signal processor (DSP) 52, a noise reduction unit 53, a memory 54, a signal processing unit 55, an image processing unit 56, a display control unit 57, a video signal generation unit 58, and the central control unit 59 are realized. The image processing unit 56 comprises an identification information assignment section 61 and a frame rate conversion section 62, and similarly, these functions are also realized by the program in the program memory being operated by the central control unit 59 composed of the image processor. In addition, the central control unit 59 receives information from the endoscope 12 and the light source device 13, and controls each unit of the processor device 14 and controls the endoscope 12 or the light source device 13, based on the received information. Further, the central control unit 59 also receives information, such as an instruction through the keyboard 16.

The image acquisition unit 51 that is a medical image acquisition unit acquires the digital image signal of the endoscope image input from the endoscope 12. The image acquisition unit 51 acquires, for each frame, an image signal obtained by imaging the observation target illuminated with each illumination light. The type of illumination light, that is, the spectrum of illumination light, is one of imaging conditions. The image acquisition unit 51 acquires a plurality of types of endoscope images with imaging conditions different from each other, such as the spectrum of illumination light.

Examples of the imaging conditions include the observation distance with the observation target, the zoom magnification of the endoscope 12, and the like, in addition to the spectrum of illumination light, that is, the light amount ratios between LED20a to 20d. The light amount ratios are acquired from the central control unit 59. The observation distance includes, for example, a non-magnified observation distance in which the observation distance is a long distance, a magnified observation distance in which the observation distance is a short distance, and the like, and is acquired by the exposure amount obtained from the endoscope image. The observation distance may be acquired by frequency analysis of the image. The zoom magnification includes, for example, non-magnification for non-magnification observation, low magnification to high magnification for magnification observation, and the like, and can be acquired based on the change operation of the zoom operation portion 12f. In the present embodiment, the spectrum of illumination light is used as the imaging condition.

The acquired image signal is transmitted to the DSP 52. The DSP 52 performs digital signal processing, such as color correction processing, on the received image signal. The noise reduction unit 53 performs noise reduction processing by, for example, a moving average method or a median filtering method, on the image signal on which the color correction processing or the like has been performed by the DSP 52. The noise-reduced image signal is stored in the memory 54.

The signal processing unit 55 acquires the noise-reduced image signal from the memory 54. Then, signal processing, such as color conversion processing, color enhancement processing, and structure enhancement processing, is performed as necessary on the acquired image signal, and a color endoscope image in which the observation target appears is generated.

In the normal observation mode or the diagnosis support observation mode, the signal processing unit 55 performs image processing for the normal observation mode, such as the color conversion processing, the color enhancement processing, and the structure enhancement processing, on the input noise-reduced image signal for the normal image for one frame. The image signal subjected to the image processing for the normal observation mode is input to the image processing unit 56 as the normal image.

In the special observation mode or the diagnosis support observation mode, the image processing for the first special observation mode, such as the color conversion processing, the color enhancement processing, and the structure enhancement processing, is performed on the input noise-reduced image signal for the first image for one frame in the first special observation mode. The image signal subjected to the image processing for the first special observation mode is input to the image processing unit 56 as the first image.

Since the endoscope image generated by the signal processing unit 55 is a normal observation image in a case where the observation mode is the normal observation mode, and is a special observation image including the first image in a case where the observation mode is the special observation mode, and the contents of the color conversion processing, the color enhancement processing, and the structure enhancement processing differ depending on the observation modes. In the case of the normal observation mode, the signal processing unit 55 performs the above-described various types of signal processing to make the observation target have a natural color tone and generates the normal observation image. In the case of the special observation mode, for example, the signal processing unit 55 performs the above-described various types of signal processing to enhance the blood vessel as the observation target and generates the special observation image including the first image.

The semiconductor light sources include the V-LED 20a that emits violet light V (first narrow band light) having a central wavelength of 410±10 nm and a wavelength range of 380 to 420 nm, and the B-LED 20b that emits blue light B (second narrow band light) having a central wavelength of 450±10 nm and a wavelength range of 420 to 500 nm. Therefore, in the first image which is the special observation image generated by the signal processing unit 55, blood vessels (so-called surface layer blood vessels) or blood at a relatively shallow position in the observation target with a surface of the mucous membrane as a reference has a magenta-based color (for example, a brown color). As a result, in the first image, the blood vessels or the bleeding (blood) of the observation target is enhanced by a difference in color with respect to the mucous membrane represented by a pink-based color.

The image processing unit 56 performs various types of image processing. The image processing unit 56 comprises the identification information assignment section 61 and the frame rate conversion section 62. The identification information assignment section 61 generates an identification information-assigned medical image in which a part of data constituting the acquired endoscope image is assigned as identification information indicating the type of the endoscope image by changing the part of the data constituting the endoscope image, or by changing the part of the data constituting the endoscope image in at least one type of the medical image and not changing the part of the data constituting the endoscope image in another type of the medical image. In a case where the identification information-assigned medical image is sent to the display 15 or the medical image processing device 17, the frame rate conversion section 62 converts the frame rate for these as necessary. In the present embodiment, since the medical image is an endoscope image, an identification information-assigned endoscope image is generated as the identification information-assigned medical image.

The data constituting the endoscope image means, for example, not data other than the image, such as a header portion of an information container storing the endoscope image, but data of the image itself. It is preferable that the data constituting the endoscope image is data of an image file that can be handled particularly in a general-purpose PC. The format or expression method of the data is not limited as long as it is data constituting the endoscope image, and a pixel value, a frequency distribution, a value calculated using these, or the like can be used.

The identification information assignment section 61 identifies the type of the endoscope image from information regarding the light emission of the light source unit 20 controlled through the light source processor 21 by the central control unit 59 in synchronization with the imaging control unit, and changes a part of data constituting the image itself of the acquired endoscope image according to the identified type of the endoscope image. Alternatively, the identification information assignment section 61 changes the part of the data constituting the image itself of the acquired endoscope image for a certain type of the endoscope image, but does not change the data constituting the image itself of the acquired endoscope image for another type of the endoscope image, according to the identified type of the endoscope image. The identification information assignment section 61 generates the identification information-assigned endoscope image by changing or not changing, in a part of the endoscope image, data constituting the image. Therefore, the identification information-assigned endoscope image includes an endoscope image in which data constituting the endoscope image is changed and an endoscope image as it is without changing the data constituting the endoscope image.

The type of the endoscope image is identified by recognizing the identification information in the identification information-assigned endoscope image. In a case where the identification information is a pixel value, correspondence information in which the position of a pixel when the pixel value is changed and a value of the changed pixel value are associated with the type of the endoscope image in the identification information-assigned endoscope image is prepared, and the corresponding type of the endoscope image is ascertained using the correspondence information for the pixel which is the identification information among the pixels constituting the identification information-assigned endoscope image. As a result, it is possible to identify which type of endoscope image the identification information-assigned endoscope image belongs to. Accordingly, in order to identify the type of the endoscope image, it is not necessary to use data other than the data of the image itself, such as the header portion.

It is preferable that the data constituting the endoscope image is a pixel value of a pixel constituting the endoscope image. In this case, the identification information assignment section 61 generates the identification information-assigned endoscope image by changing the pixel values of a predetermined part of the pixels constituting the endoscope image according to the identified type of the endoscope image. In addition, in some cases, in order to identify the type of the endoscope image in which the pixel value is changed, another type of endoscope image in which the pixel value is not changed is used as the identification information-assigned endoscope image.

It is preferable that the pixel value to be changed is changed so as not to affect, for example, a case where the endoscope image is used for observation, diagnosis, or the like. Any of color information or brightness information can also be used as the pixel value. Further, it is possible to perform a change such that a user cannot visually recognize the pixel of which the pixel value has been changed, a change in an aspect that can be visually recognized by the user but does not affect the visual recognition of the observation target or the like appearing in the endoscope image, or the like.

Examples of the case where the user cannot visually recognize the pixel of which the pixel value has been changed include methods such as changing the pixel values of a part of pixels of the endoscope image to specific pixel values so as not to affect the user's visual recognition, changing the pixel values of a part of pixels of the endoscope image to dummy pixel values, or applying a digital watermark to the endoscope image. The digital watermark can also be applied to data constituting the endoscope image other than the pixel value.

In a case where a part of pixels of the endoscope image are changed to pixels having specific pixel values so as not to affect the user's visual recognition, it is possible to perform a change such as an increase or decrease in at least one or a plurality of values of red, green, or blue constituting the pixel values at a part of positions of the endoscope image or in brightness information. In correspondence with the type of the endoscope image, any one of red, green, blue, or brightness may be changed or the change of the pixel value may be increased or decreased. Any method can be employed as long as the pixel of which the pixel value has been changed cannot be visually recognized in a case where the user visually recognizes the pixel. In this case, the type of the endoscope image can be identified by, for example, comparing the changed pixel value with surrounding pixel values.

Even in a case where the pixel value of a part of the endoscope image is changed by being replaced with the dummy pixel value, the pixel value can be replaced with the dummy pixel value without affecting the user's visual recognition. The dummy pixel value is, for example, a pixel value decided on in advance according to the type of the endoscope image. The dummy pixel value is replaced with a pixel value at a predetermined position of the endoscope image. In this case, the type of the endoscope image can be identified by acquiring the dummy pixel value at the replaced position.

In a case where the digital watermark is applied to the endoscope image, a known digital watermark technique can be employed. For example, by embedding watermark information including the type of the endoscope image into the endoscope image, it is possible to identify the type of the endoscope image, for example, in a case where the watermark information is acquired or the watermarked endoscope image is restored.

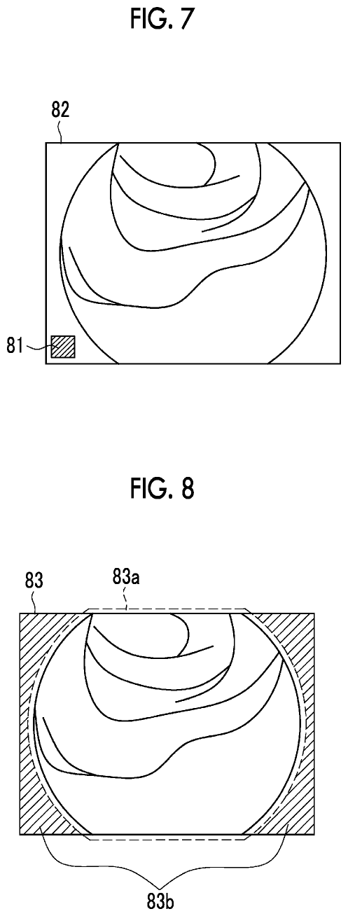

As shown in FIG. 7, an identification information-assigned endoscope image 82 is generated by changing a part of data constituting the endoscope image to identification information 81 which is a preset pixel value. In the identification information-assigned endoscope image 82, the identification information 81 is assigned to a region that is not a portion of the observation target appearing in the identification information-assigned endoscope image 82, and is assigned in an aspect that can be visually recognized by the user but does not affect the visual recognition of the observation target or the like appearing in the endoscope image.

It is preferable that the data constituting the endoscope image is data constituting a preset region of the endoscope image. Therefore, it is preferable that the identification information of the identification information-assigned endoscope image is in a preset region of the endoscope image. Examples of the preset region include a mask portion where the observation target does not appears, an edge portion of a region where the observation target appears, and the like in the endoscope image. As shown in FIG. 8, in the present specification, an endoscope image 83 indicates the entire image appearing on the display 15, and refers to an image including an observation target portion 83a and a mask portion 83b. In FIG. 8, the observation target portion 83a is indicated by being surrounded by a broken line, and the mask portion 83b is indicated by adding diagonal lines. In the case of FIG. 7, the identification information 81 is changed to a predetermined pixel value in a state in which a part of the region of the mask portion 83b of the endoscope image can be visually recognized by the user.

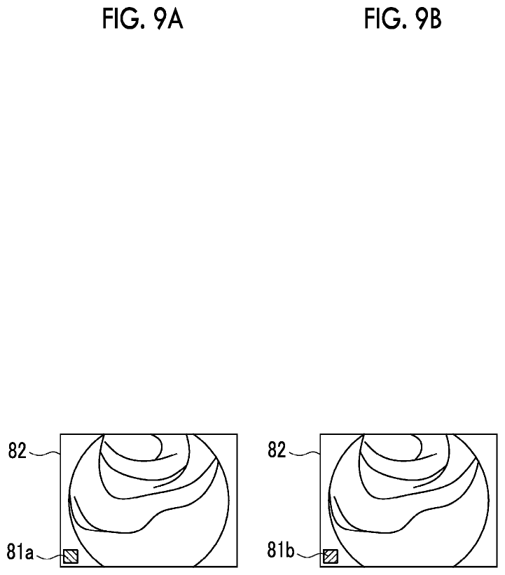

As shown in FIGS. 9A and 9B, the identification information assignment section 61 performs a change by replacing a pixel value with another pixel value, which is different according to the type of the endoscope image, at the same position in the endoscope image 83, thereby assigning the changed pixel value as the identification information. In FIG. 9A, first identification information 81a is assigned, and in FIG. 9B, second identification information 81b is assigned. Therefore, it is possible to identify that the identification information-assigned endoscope image 82 of FIG. 9A and the identification information-assigned endoscope image 82 of FIG. 9B are different types of endoscope images, and it is possible to identify the specific type of the endoscope image of each of the identification information-assigned endoscope images 82, only from the image data of the identification information-assigned endoscope image 82 without relying on information, such as the header portion other than the image data, or separate information synchronously sent from, for example, the central control unit 59 or the light source processor 21. Further, by making the first identification information 81a and the second identification information 81b different colors or the like that can be identified by the user, the user can correctly grasp the type of the endoscope image only by viewing the identification information-assigned endoscope image 82 at a glance.

It is preferable that the plurality of types of endoscope images include a display image for display on the display 15 and an analysis image for analysis related to diagnosis support information. By including two types of endoscope images as the types of endoscope images, that is, the display image and the analysis image, for example, the display image can be used as an image to be displayed on the display 15, and a type of endoscope image that yields a good analysis result in a case of being used as a target of image analysis using machine learning or the like even though it is difficult to recognize by the user when visually recognized can be used as an image to be subjected to the image analysis. In this case, the analysis image can be set not to be displayed on the display 15.

In the present embodiment, in the diagnosis support observation mode, two types of endoscope images having different spectra of illumination light and different types between the normal image and the first image are automatically acquired. Therefore, the normal image is used as the display image, and the first image is used as the analysis image.

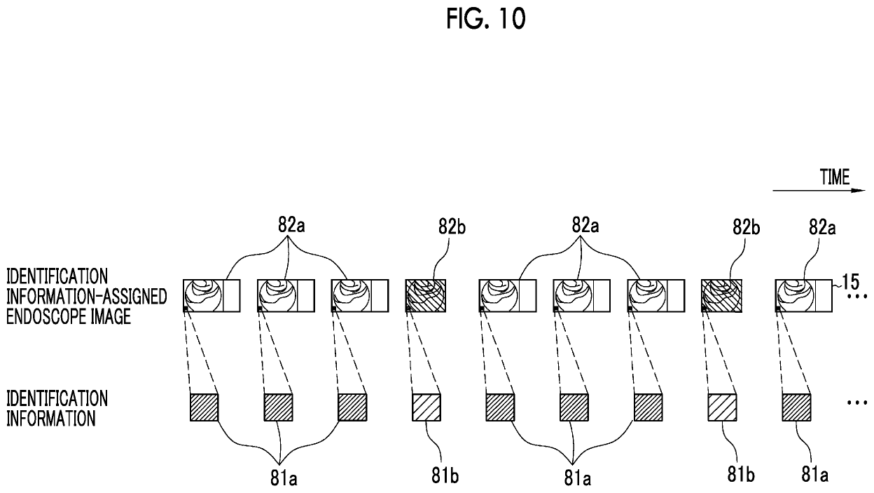

As shown in FIG. 10, in the present embodiment, a pattern in which the normal image 71 is acquired for three frames and then the first image 72 is acquired for one frame is repeated (see FIG. 6). The identification information assignment section 61 assigns the identification information 81 to each of the acquired endoscope images to generate the identification information-assigned endoscope image 82. Therefore, the identification information assignment section 61 assigns the first identification information 81a for identifying the normal image to the normal image 71 by changing the pixel value of the pixel of a predetermined region of the mask portion of the endoscope image to a predetermined pixel value, thereby generating the first identification information-assigned endoscope image 82a. The second identification information 81b for identifying the first image is similarly assigned to the first image 72, and a second identification information-assigned endoscope image 82b is generated. In FIG. 10, the normal image 71 is shown by the normal image 71 and the display 15, which indicates that the normal image 71 is displayed on the display 15. Since the first image 72 is not displayed on the display 15, it is shown as it is. In addition, in the drawing, different shading for the identification information 81 indicates different pieces of identification information 81.

In FIG. 10, as described above, since the normal image 71 and the first image 72 are seen to have different color tones when viewed by a person, diagonal lines are added to the first image 72 to indicate a visual difference between the two endoscope images. Further, the identification information 81 assigned to the identification information-assigned endoscope image 82 is enlarged and shown. In the drawing, in order to avoid complication of the drawing, reference numerals may be added to only a part thereof.

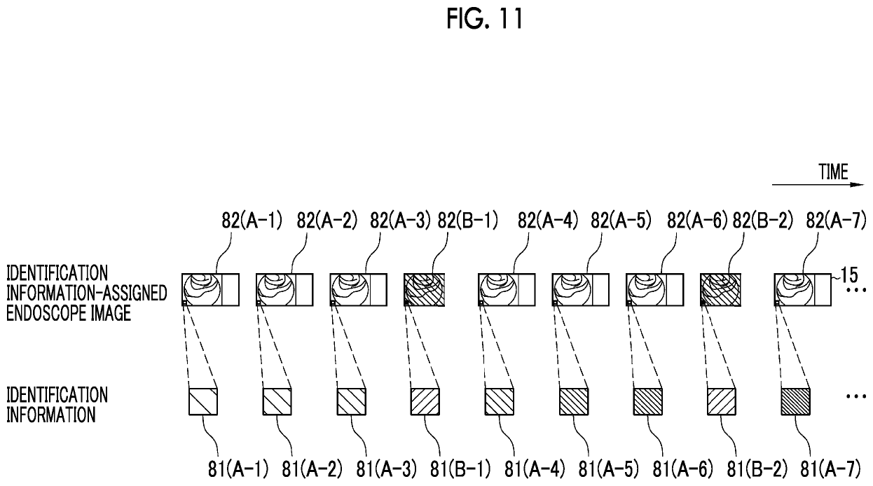

Further, the identification information 81 may include two or more pieces of information. For example, the identification information 81 may include information regarding an imaging order in addition to the type of the endoscope image. As shown in FIG. 11, as in the case of FIG. 10, a pattern in which the normal image 71 is acquired for three frames and then the first image 72 is acquired for one frame is repeated (see FIG. 6). The identification information assignment section 61 assigns the identification information 81 to each of the acquired endoscope images to generate the identification information-assigned endoscope image 82. Here, the identification information assignment section 61 assigns first identification information 81 (A-1) for identifying that the image is the normal image and that has been captured first is assigned to a first frame of the normal image 71 by changing the pixel value of the pixel of a predetermined region of the mask portion of the endoscope image to the predetermined pixel value, thereby generating a first identification information-assigned endoscope image 82 (A-1). The first identification information 81 (A-1) indicates identification information (A-1) indicating that the image is the normal image and that is captured first in the imaging order.

First identification information 81 (A-2) for identifying that the image is the normal image and that has been captured second is assigned to a next frame of the normal image 71 by changing the pixel value of the pixel of the predetermined region of the mask portion of the endoscope image to a predetermined pixel value, and a first identification information-assigned endoscope image 82 (A-2) is generated. The first identification information 81 (A-2) indicates identification information (A-2) indicating that the image is the normal image and that is captured second in the imaging order. Similarly, FIG. 11 shows that the first identification information-assigned endoscope images 82 in which the first identification information 81 (A-3) to the first identification information (A-7) are assigned are generated.

Similarly to the normal image 71, second identification information 81 (B-1) for identifying that the image is the first image and that has been captured first is assigned to a first frame of the first image 72 by changing the pixel value of the pixel of the predetermined region of the mask portion of the endoscope image to a predetermined pixel value, and a second identification information-assigned endoscope image 82 (B-1) is generated. The second identification information 81 (B-1) indicates identification information (B-1) indicating that the image is the first image and that is captured first in the imaging order.

Second identification information 81 (B-2) for identifying that the image is the first image and that has been captured second is assigned to a next frame of the first image 72 by changing the pixel value of the pixel of the predetermined region of the mask portion of the endoscope image to a predetermined pixel value, and a second identification information-assigned endoscope image 82 (B-2) is generated. The second identification information 81 (B-2) indicates identification information (B-2) indicating that the image is the first image and that is captured second in the imaging order. FIG. 11 shows that the second identification information-assigned endoscope images 82 in which the second identification information 81 (B-1) and the second identification information (B-2) are assigned are generated.

It is preferable that the identification information 81 also includes information regarding the imaging order in addition to the type of the endoscope image because the information can be easily obtained only from the image data of the endoscope image.

Further, the identification information assignment section 61 may assign the identification information to the analysis image by changing a part of data constituting the analysis image and may assign the identification information to the display image by not changing a portion of data constituting the display image, which corresponds to the data assigned as the identification information in the analysis image. Similarly, the identification information may be assigned to the display image by changing a part of data constituting the display image, and the identification information may be assigned to the analysis image by not changing a portion of data constituting the analysis image, which corresponds to the data assigned as the identification information in the display image.

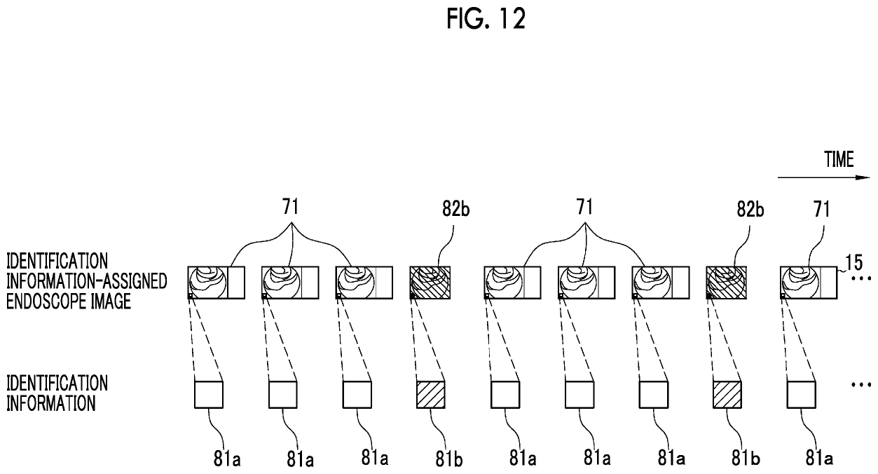

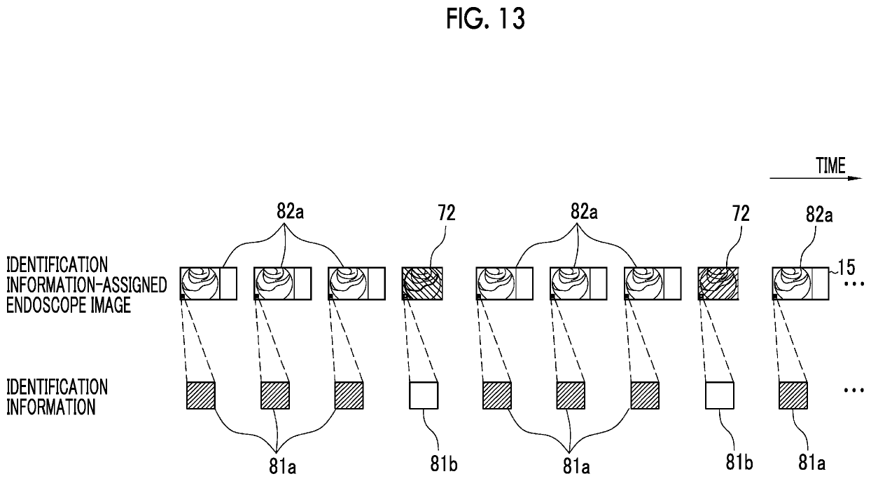

As shown in FIG. 12, the identification information assignment section 61 assigns the second identification information 81b to the first image 72, which is the analysis image, by changing a part of data constituting the first image 72, thereby generating the second identification information-assigned endoscope image 82b, and assigns the first identification information 81a to the normal image 71, which is the display image, without changing a portion of data constituting the normal image 71, which corresponds to the data assigned as the second identification information 81b in the first image 72, thereby generating the first identification information-assigned endoscope image 82a. In FIG. 12, the first identification information 81a is shown without shading, which indicates that the data constituting the original endoscope image is not changed by the identification information assignment section 61. The same applies to FIG. 13.

As shown in FIG. 13, the identification information assignment section 61 assigns the first identification information 81a to the normal image 71, which is the display image, by changing a part of data constituting the normal image 71, thereby generating the first identification information-assigned endoscope image 82a, and assigns the second identification information 81b to the first image 72, which is the analysis image, without changing a portion of data constituting the first image 72, which corresponds to the data assigned as the first identification information 81a in the normal image 71, thereby generating the second identification information-assigned endoscope image 82b.

As described above, in a case where the identification information assignment section 61 assigns the identification information 81 to the analysis image or the display image, the normal image, which is the display image, and the first image, which is the analysis image, can be identified based on whether or not a part of data constituting the image is changed. Therefore, for example, in a case where there are two types of endoscope images, it is sufficient to change a part of data in one type of endoscope image, which makes it possible to reduce the time and effort for assigning the identification information 81. Further, in a case where the identification information 81 is assigned by changing a part of data only in an identification image, the image data of the display image is not changed at all. Accordingly, in a case where the display image is displayed on the display 15 or the like, the identification information 81 does not affect the visibility of the user at all, which is preferable.

The identification information-assigned endoscope image 82 is sent from the processor device 14 to the medical image processing device 17. The medical image processing device 17 receives the identification information-assigned endoscope image 82 transmitted from the processor device 14 and performs control to display the identification information-assigned endoscope image 82 on the display 15 based on the type of the identification information-assigned endoscope image 82. Further, the medical image processing device 17 performs display processing or analysis processing according to the type of the identification information-assigned endoscope image 82. After the analysis processing, an analysis result image, which shows the analysis result through display, is transmitted to the processor device 14. Further, a superimposition image is generated by superimposing the analysis result image on the identification information-assigned endoscope image 82, and the superimposition image is displayed on the display 15.

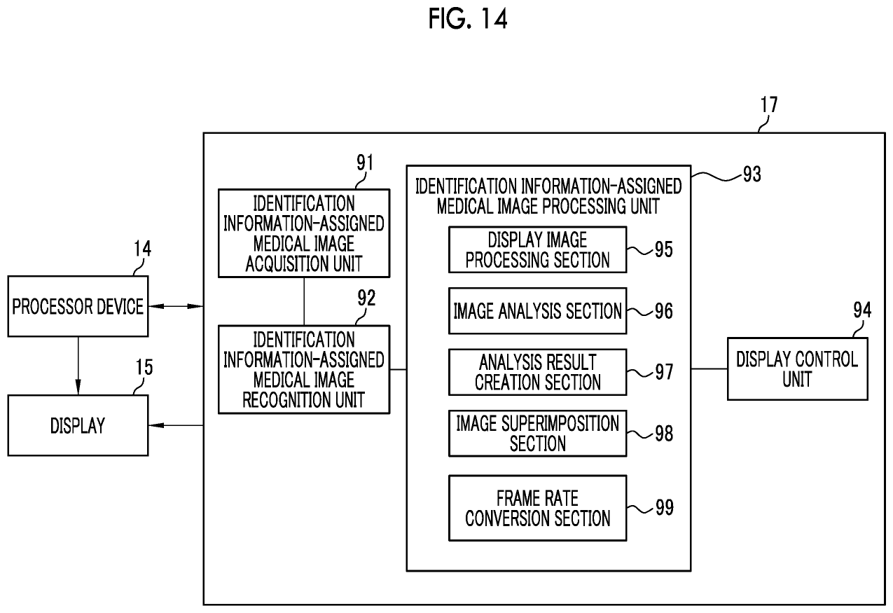

The medical image processing device 17 is a general-purpose PC provided with a processor and exhibits various functions by installing software. In the medical image processing device 17, similarly to the processor device 14, a program related to processing, such as image analysis processing, is stored in a program memory. In the medical image processing device 17, the functions of an identification information-assigned medical image acquisition unit 91, an identification information-assigned medical image recognition unit 92, an identification information-assigned medical image processing unit 93, and a display control unit 94 (see FIG. 14) are realized by the program in the program memory being operated by the central control unit composed of an image processor, which is a second processor, or the like. The identification information-assigned medical image processing unit 93 comprises a display image processing section 95, an image analysis section 96, an analysis result creation section 97, an image superimposition section 98, and a frame rate conversion section 99 (see FIG. 14), and similarly, these functions are also realized by the program in the program memory being operated by the central control unit composed of the image processor. Further, the central control unit receives information from the processor device 14 or the like and controls each unit of the medical image processing device 17 based on the received information. In addition, the central control unit is connected to a user interface, such as a keyboard (not shown), and receives information, such as an instruction through the user interface.

Further, the medical image processing device 17 is connected to the display 15 and displays various images generated by the medical image processing device 17. Various devices may be connected to the medical image processing device 17. Examples of the various devices include a user interface, such as a keyboard for issuing an instruction, and a storage for storing data, such as an image.

As shown in FIG. 14, the medical image processing device 17 comprises the identification information-assigned medical image acquisition unit 91, the identification information-assigned medical image recognition unit 92, the identification information-assigned medical image processing unit 93, and the display control unit 94. The identification information-assigned medical image acquisition unit 91 acquires a plurality of types of identification information-assigned endoscope images 82 sent from the processor device 14. The acquired images are sent to the identification information-assigned medical image recognition unit 92. In the identification information-assigned endoscope image 82, a part of data constituting the endoscope image is assigned as the identification information 81. The identification information-assigned medical image recognition unit 92 recognizes the type of the identification information-assigned endoscope image 82 based on the identification information 81 assigned to the identification information-assigned endoscope image 82. The identification information-assigned medical image processing unit 93 controls display on the display 15 based on the type of the identification information-assigned endoscope image 82 and performs image processing set for each type of the identification information-assigned endoscope image 82 on the identification information-assigned endoscope image 82.

The identification information-assigned medical image recognition unit 92 recognizes the type of the identification information-assigned endoscope image 82 based on the identification information of the identification information-assigned endoscope image 82. The type of the identification information-assigned endoscope image 82 is the same as the type of the endoscope image that is the source of the identification information-assigned endoscope image 82. The recognition is performed based on the content of the identification information 81. The identification information-assigned medical image recognition unit 92 is provided with, in advance, correspondence information in which the content of the identification information 81 and the type of the endoscope image correspond to each other. Based on the correspondence information and the content of the identification information 81 included in the identification information-assigned endoscope image 82, which type of endoscope image the identification information-assigned endoscope image 82 belongs to is identified. The identification information 81, the content of the identification information 81, and the like are the same as the identification information 81 assigned by the identification information assignment section 61 in the processor device 14 and are as described above.

As shown in FIG. 14, the identification information-assigned medical image processing unit 93 comprises the display image processing section 95, the image analysis section 96, the analysis result creation section 97, the image superimposition section 98, and the frame rate conversion section 99.

The image processing performed by the identification information-assigned medical image processing unit 93 includes the display image processing and the analysis image processing. It is preferable that the plurality of types of identification information-assigned endoscope images 82 include the display image for display on the display 15 and the analysis image for analysis related to the diagnosis support information. In addition, it is preferable that the identification information-assigned medical image processing unit 93 performs, in a case where the identification information-assigned endoscope image 82 is the display image, the display image processing on the display image, and performs, in a case where the identification information-assigned endoscope image 82 is the analysis image, the analysis image processing on the analysis image.

In a case where the type of the identification information-assigned endoscope image 82 is the display image that is the type of the endoscope image for display on the display 15, the display image processing section 95 performs the display image processing on this image. It is preferable that the display image processing is different for each type of the identification information-assigned endoscope image 82. An image suitable for display on the display 15 is generated by the display image processing performed by the display image processing section 95.

In a case where the type of the identification information-assigned endoscope image 82 is the analysis image that is the type of the endoscope image for analysis related to the diagnosis support information, the image analysis section 96 performs the analysis image processing on this image. It is preferable that the analysis image processing is different for each type of the identification information-assigned endoscope image 82, and it is preferable that the analysis image processing is different for each content of analysis. The diagnosis support information can be obtained by the image analysis processing performed by the image analysis section 96. The diagnosis support information is presented to the user through the analysis result image or the like showing the diagnosis support information.

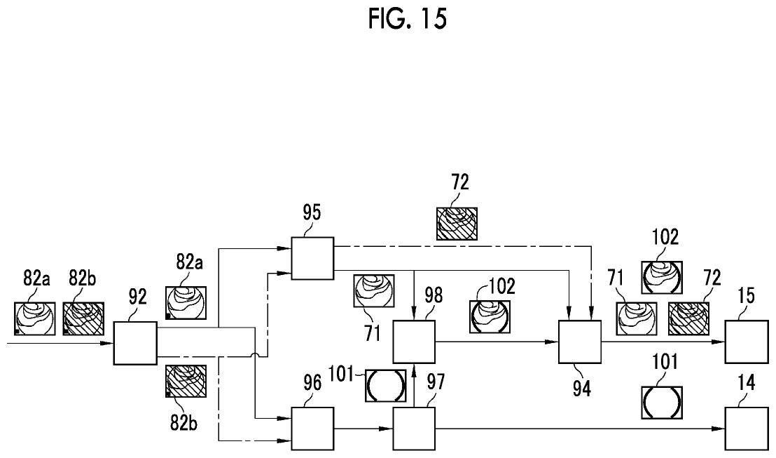

As shown in FIG. 15, in the present embodiment, specifically, since the display image is the normal image 71 and the analysis image is the first image 72, the identification information-assigned endoscope image 82 includes two types: the first identification information-assigned endoscope image 82a in which the first identification information 81a is assigned to the normal image 71; and the second identification information-assigned endoscope image 82b in which the second identification information 81b is assigned to the first image 72. In the identification information-assigned endoscope image 82, the identification information 81 is read by the identification information-assigned medical image recognition unit 92, and the type of the endoscope image is specified. The identification information or the identification information-assigned endoscope image is referred to as, for example, the identification information 81 or the identification information-assigned endoscope image 82 in a case where the types are not distinguished.

After the type is specified in each of the identification information-assigned endoscope images 82, the processing for the first identification information-assigned endoscope image 82a and the processing for the second identification information-assigned endoscope image 82b are carried out through separate flows, respectively.

The first identification information-assigned endoscope image 82a is sent to the display image processing section 95 and the image analysis section 96. The display image processing section 95 performs image processing for display on the display 15. The image analysis section 96 performs analysis on the first identification information-assigned endoscope image 82a as a target as necessary. The second identification information-assigned endoscope image 82b is sent to the display image processing section 95 and the image analysis section 96. In a case where the second identification information-assigned endoscope image 82b is displayed on the display, the display image processing section 95 performs image processing for displaying the second identification information-assigned endoscope image 82b on the display 15. The image analysis section 96 performs analysis on the second identification information-assigned endoscope image 82b as a target as necessary. In FIG. 15, in order to distinguish the flow of the first identification information-assigned endoscope image 82a and the flow of the second identification information-assigned endoscope image 82b, the flow of the second identification information-assigned endoscope image 82b is indicated by an alternating long-dash and short-dash line.

In the present embodiment, the identification information-assigned medical image processing unit 93 performs the display image processing through the display image processing section 95 because the first identification information-assigned endoscope image 82a is the display image and does not perform image analysis, and performs the analysis image processing through the image analysis section 96 because the second identification information-assigned endoscope image 82b is the analysis image and does not perform display image processing because the second identification information-assigned endoscope image 82b is not displayed on the display 15.

The image analysis section 96 performs the analysis image processing for computer-aided diagnosis (CAD) on the identification information-assigned endoscope image 82 as a target. As the analysis image processing, known analysis image processing can be performed. Through the analysis image processing based on the endoscope image, the diagnosis support information, such as various feature amounts such as oxygen saturation, detection of blood vessel positions or lesion positions, or estimation of lesion stages, is output.

It is preferable that the image analysis section 96 performs the analysis image processing using a machine learning-based analysis model. It is preferable for the machine learning-based analysis model to use a convolutional neural network that outputs a good result in image analysis. Further, it is preferable that the analysis model is different for each type of the identification information-assigned endoscope image 82. This is because the content of the analysis image processing capable of outputting a good result is different for each type of the identification information-assigned endoscope image 82. For the same reason, it is preferable that the analysis model is different for each analysis content. Therefore, it is preferable that the image analysis section 96 comprises a plurality of the analysis models and uses an appropriate analysis model according to the type of the endoscope image. It is preferable that the plurality of analysis models generate different pieces of diagnosis support information as the analysis result.

In the present embodiment, the analysis image processing by the image analysis section 96 is performed on the second identification information-assigned endoscope image 82b. Since the second identification information-assigned endoscope image 82b is the first image 72, a surface layer result or the like is enhanced, and a good result can be obtained by the analysis model for distinguishing between a neoplastic polyp and a non-neoplastic polyp. Therefore, the image analysis section 96 analyzes the second identification information-assigned endoscope image 82b using the analysis model for detecting a neoplastic polyp and generates an analysis result. This analysis model distinguishes between a neoplastic polyp and a non-neoplastic polyp, and even in a case where there is a non-neoplastic polyp, the analysis model does not notify or alert the user unless the polyp is a neoplastic polyp.

It is preferable that the identification information-assigned medical image processing unit 93 creates the analysis result image indicating the result of the analysis image processing and generates the analysis image by superimposing the analysis result image on the display image. Specifically, the analysis result creation section 97 can acquire the analysis result by the image analysis section 96 and create the analysis result in a form capable of notifying the user of the analysis result by the image analysis section 96, for example, in a form of sound, an image, or the like. In the present embodiment, the user is notified of whether or not there is a neoplastic polyp by displaying, on the display 15, a frame or a frame color displayed on an edge portion of a region where the observation target appears in the endoscope image. A red frame is displayed in a case where there is a neoplastic polyp, and a green frame is displayed in a case where there is no neoplastic polyp. In the present embodiment, since a neoplastic polyp is not detected, the analysis result creation section 97 generates, as the analysis result, an analysis result image 101 of a green frame displayed at the edge portion of the region where the observation target appears.

The image superimposition section 98 acquires, from the display image processing section 95, the endoscope image on which the analysis result image 101 is superimposed. As the endoscope image on which the analysis result image 101 is superimposed, the normal image 71 obtained by performing the display image processing on the first identification information-assigned endoscope image 82a is used because it is preferable to use the display image. Further, the analysis result image 101 is acquired from the analysis result creation section 97. Then, a superimposition image 102 in which the analysis result image 101 is superimposed on the normal image 71 acquired from the display image processing section 95 is generated. The generated superimposition image 102 is transmitted to the display control unit 94.

The display control unit 94 acquires three types of images from the display image processing section 95 and the image superimposition section 98 and performs control to display the images on the display 15. The normal image 71 based on the first identification information-assigned endoscope image 82a and the first image 72 based on the second identification information-assigned endoscope image 82b are acquired from the display image processing section 95. The superimposition image 102 is acquired from the image superimposition section 98. Therefore, the display control unit 94 performs control to display the normal image 71, the first image 72, and/or the superimposition image 102 on the display 15 in response to an instruction. As described above, by connecting the medical image processing device 17 to the display 15, one or a plurality of these images can be displayed in a preset layout.

Further, the analysis result image 101 created by the analysis result creation section 97 may be sent to the processor device 14. In this case, in the processor device 14, the analysis result image 101 can be superimposed on various endoscope images. In addition, the superimposed image can also be displayed on the display 15 connected to the processor device 14. As described above, it is desirable to send the analysis result image 101 to the processor device 14 because the availability of the analysis result image is increased.

Although a case where the first image 72, which is the analysis image, is not displayed on the display 15 has been described in the above-described embodiment, the first image 72 may be displayed on the display 15 depending on the type of the analysis image. The analysis image to be displayed is preferably an analysis image that is a type of endoscope image which is observable and for which diagnostic utility using the endoscope image has been established. For example, since an analysis image, which is an endoscope image or the like obtained through special light observation using blue narrow-band light, such as the first image 72, has established diagnostic utility using this type of endoscope image and is helpful in diagnosis when viewed by a doctor, the analysis image is displayed on the display 15. Examples of the type of analysis image that is preferably displayed include an analysis image using blue narrow-band light, an endoscope image subjected to color enhancement processing or structure enhancement processing, and an analysis image displaying biological information such as an oxygen saturation.

On the other hand, as the analysis image, an analysis image that is difficult to observe and for which diagnostic utility using that type of endoscope image has not yet been established may not be displayed on the display 15. For example, an endoscope image or the like using only the violet light V as illumination light is an analysis image helpful for analysis of oxygen saturation or the like, but in some cases, displaying the endoscope image or the like on the display 15 may not be helpful for diagnosis. Therefore, the identification information-assigned medical image processing unit 93 may perform control to display the identification information-assigned endoscope image 82 on the display 15 based on the type of the identification information-assigned endoscope image 82.

In a case where the analysis image is displayed on the display 15, it is preferable to display the analysis image, for example, on a sub screen of the display 15. In this case, the display 15 includes a main screen and the sub screen. Therefore, the identification information-assigned medical image processing unit 93 displays the display image on the main screen of the display 15. Further, it is preferable that whether or not to display the analysis image on the sub screen of the display 15 is decided on based on the type of the identification information-assigned endoscope image 82, and that the identification information-assigned endoscope image 82 which is decided to be displayed is displayed on the sub screen of the display 15.

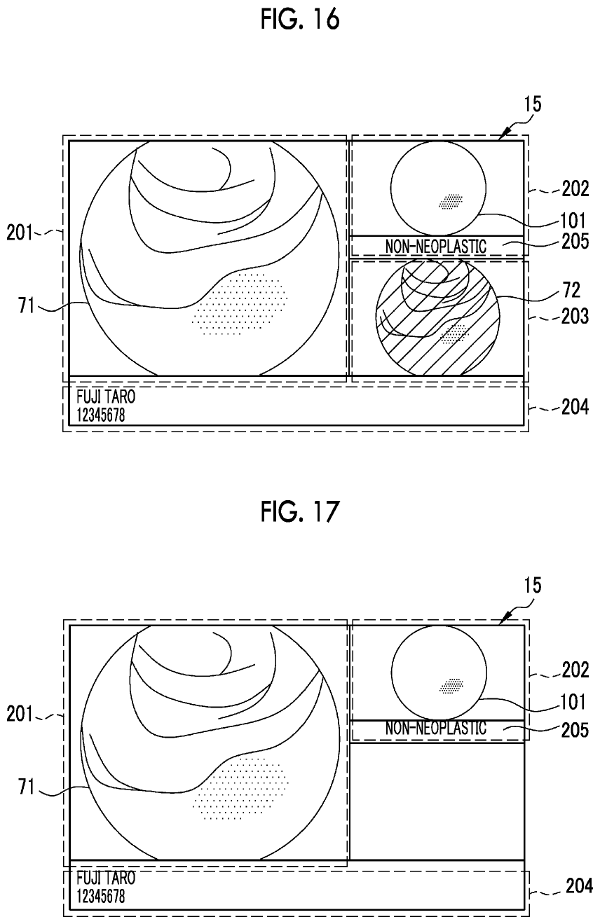

As shown in FIG. 16, in the present embodiment, the display 15 includes one main screen 201 and two sub screens, that is, a first sub screen 202 and a second sub screen 203. In addition, the display 15 includes a patient information display screen 204 for displaying patient information. The normal image 71 that is the display image is displayed on the main screen 201. For example, the analysis result image 101 created by the analysis result creation section 97 is displayed on the first sub screen 202. In FIG. 16, the analysis result image is an image in which a distinction result of a region of interest that is the analysis result is displayed in a map format. The distinction result displayed in the map format indicates the distinction result distinctively by the color of the region of interest in the map, for example. Further, the first sub screen 202 includes an analysis result text display screen 205 for displaying the analysis result by text. In the analysis result text display screen 205, for example, the analysis result is shown in text by displaying, for example, “NON-NEOPLASTIC” or “HYPERPLASTIC”.

For example, the endoscope image used by the analysis result creation section 97 to create the analysis result image 101 among the analysis images is displayed on the second sub screen 203. In the present embodiment, the first image 72 that is the analysis image is displayed on the second sub screen 203. Since the type of the first image 72 is an endoscope image obtained through special light observation using blue narrow-band light, the first image 72 is helpful for diagnosis of a doctor or the like by being displayed on the second sub screen 203.

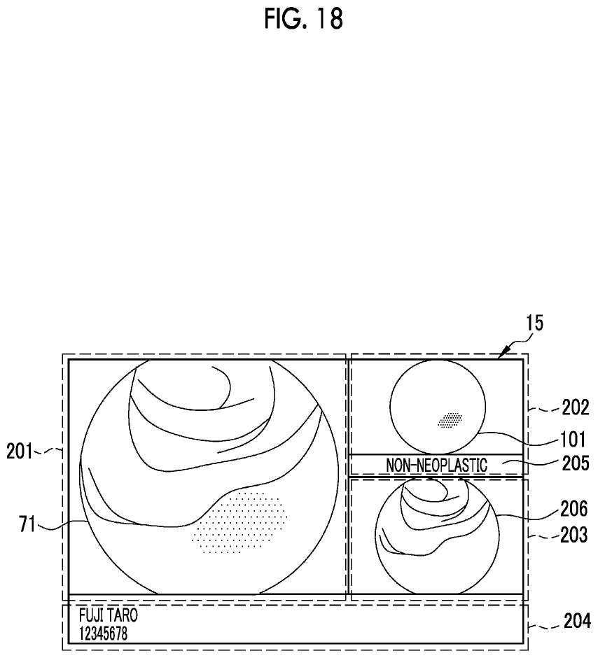

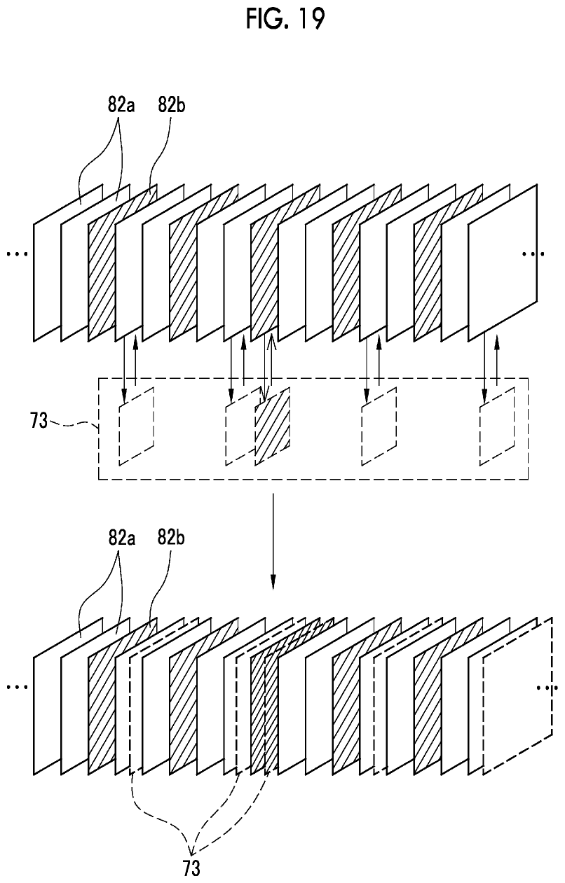

As shown in FIG. 17, in a case where the display 15 includes the sub screen, the analysis image is not displayed on the second sub screen 203 depending on the type of the analysis image. As shown in FIG. 18, in a case where the analysis image is not displayed on the second sub screen 203, a past image 206 which is an endoscope image acquired in the past for a subject of the display image displayed on the main screen 201 may be displayed. This is because there is a case where it is helpful for diagnosis or the like by comparing the past with the present in the same subject.

As described above, in a medical image processing system 18 including the processor device 14 and the medical image processing device 17, the identification information-assigned medical image acquisition unit 91 of the medical image processing device 17 acquires the identification information-assigned endoscope image 82 generated by the identification information assignment section 61 of the processor device 14. The identification information-assigned endoscope image 82 is generated by assigning a part of data constituting the endoscope image as the identification information 81 indicating the type of the endoscope image. Therefore, it is easy to identify the type of the endoscope image. For example, since the type of the endoscope image is included in the data of the endoscope image itself, the type can be easily identified in the general-purpose PC. Further, in a case where different image processing is performed by CAD or the like depending on the type of the endoscope image, the identification of the type of the endoscope image and the image processing corresponding to the type of the endoscope image can be automatically performed continuously. Therefore, in a case where the type of the endoscope image to be acquired and the image processing thereof are set for each observation mode, the burden on the user is reduced as compared with a case where the acquisition of the type of a specific endoscope image and the image processing thereof are manually switched.

The processor device 14, the medical image processing device 17, and the medical image processing system 18 are useful in a case where the type of illumination light is automatically switched in the IEE. That is, in a case where the difference in the spectrum of the illumination light is used as an imaging condition and is made to correspond to the type of endoscope image, the type of illumination light is set to be automatically switched, whereby it is possible to automatically obtain a plurality of display images and the diagnosis support information. In addition, since the processing of the display image and the processing of the analysis result can be performed by the plurality of devices, that is, the processor device 14 and the medical image processing device 17, it is possible to create or process the image with a high degree of freedom to a preferable aspect for display on the display 15, a preferable aspect for creating a medical chart or an examination report, or the like.

Further, in a case where the identification information-assigned endoscope image 82 includes a plurality of types of identification information 81, other information can be obtained in addition to the type of the endoscope image such as the spectrum of illumination light. Examples of other information include information on the imaging order.

It is preferable that the processor device 14 and the medical image processing device 17 comprise the frame rate conversion section 62 or 99 that adjusts the frame rate of the endoscope image. In the processor device 14, the frame rate conversion section 62 adjusts the frame rate of the image to be transmitted, in order to send the image to the medical image processing device 17 or to display the image such as the display image on the display 15. Similarly, in the medical image processing device 17, in a case where it is necessary, the frame rate conversion section 99 adjusts the frame rate of the image to be transmitted at the time of transmission and adjusts the frame rate of the image to be transmitted, in order to display the image such as the display image on the display 15.

In a case where the identification information-assigned endoscope image 82 including the display image and the analysis image is transmitted from the processor device 14 to the medical image processing device 17, it is preferable to adjust the frames in which the display image and the analysis image are acquired by complementing the frames with the display image and the analysis image, respectively, and to transmit the identification information-assigned endoscope image 82 at a frame rate suitable for processing of the medical image processing device 17. In a case of complementing the frames with the display image and the analysis image, for example, complementary frame images obtained by duplicating frames in which the display image and the analysis image are acquired are created, and the complementary frame image can be used as the display image or the analysis image.

The frame rate conversion section 62 in the processor device 14 creates complementary frame images 73 for the frame of the display image and the frame of the analysis image. The first identification information-assigned endoscope image 82a generated from the normal image 71, the second identification information-assigned endoscope image 82b generated from the first image 72, and the complementary frame images 73 duplicated from the first identification information-assigned endoscope image 82a and from the second identification information-assigned endoscope image 82b are combined to obtain 60 frames per second (60 fps) and then sent to the medical image processing device 17. This makes it possible for the medical image processing device 17 to acquire a video with a consistently adjusted frame rate.

As shown in FIG. 19, for example, the processor device 14 acquires the first identification information-assigned endoscope image 82a, which is the display image, for 30 fps per second and acquires the second identification information-assigned endoscope image 82b, which is the analysis image, for 15 fps per second. Diagonal lines are added to the second identification information-assigned endoscope image 82b. In order to obtain 60 fps by combining the first identification information-assigned endoscope image 82a, the second identification information-assigned endoscope image 82b, and the complementary frame image 73, the frame rate conversion section 62 obtains the complementary frame images 73 by duplicating, for the complementary frame images 73 for 15 fps, the first identification information-assigned endoscope image 82a for 10 fps and the second identification information-assigned endoscope image 82b for 5 fps. That is, in the first identification information-assigned endoscope image 82a for 30 fps, one frame out of three frames is duplicated to obtain the complementary frame image 73 for 10 fps. Similarly, in the second identification information-assigned endoscope image 82b for 15 fps, one frame out of five frames is duplicated to obtain the complementary frame image 73 for 5 fps. The complementary frame image 73 is indicated by a dotted line. In the case of duplication, the image of the frame just before the timing of the duplication can be duplicated. In this way, in a case where the first identification information-assigned endoscope image 82a is acquired at fps and the second identification information-assigned endoscope image 82b is acquired at fps, the frame rate conversion section 62 can add the complementary frame image 73 for 15 fps to obtain 60 fps.

In the processor device 14, in a case where the image acquisition unit 51 acquires the display image and the analysis image and the analysis image is not displayed on the display 15, it is possible to display an endoscope image that is easily visually recognized by complementing a frame, in which the analysis image is acquired, with the display image. The same applies to the medical image processing device 17, and it is preferable to adjust the frame rate in the image desired to be displayed on the display 15.

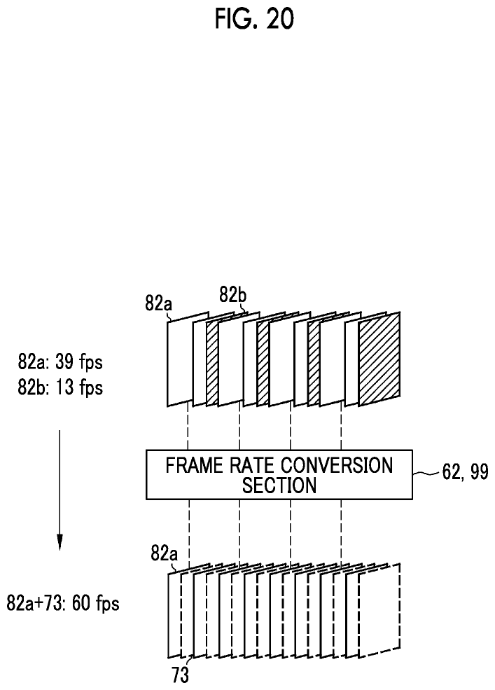

As shown in FIG. 20, for example, the processor device 14 acquires the first identification information-assigned endoscope image 82a, which is the display image, at 39 frames per second (39 fps) and acquires the second identification information-assigned endoscope image 82b, which is the analysis image, at 13 frames per second (13 fps). In a case where the analysis image is not displayed on the display 15, the frame rate conversion section 62 creates the complementary frame image 73 in each of all the frames of the display image and combines and displays the first identification information-assigned endoscope image 82a and the complementary frame image 73 on the display 15 at 60 frames per second (60 fps). As a result, good visibility of the image displayed on the display 15 can be obtained. The frame rate conversion section 99 in the medical image processing device 17 also functions in the same manner.

In the image processing unit 56, in the identification information assignment section 61 and the frame rate conversion section 62, the identification information assignment section 61 may assign the identification information 81 after the frame rate conversion section 62 performs frame rate conversion. In this case, after the frame rate conversion section 62 generates the complementary frame image 73, the identification information assignment section 61 assigns the identification information 81.

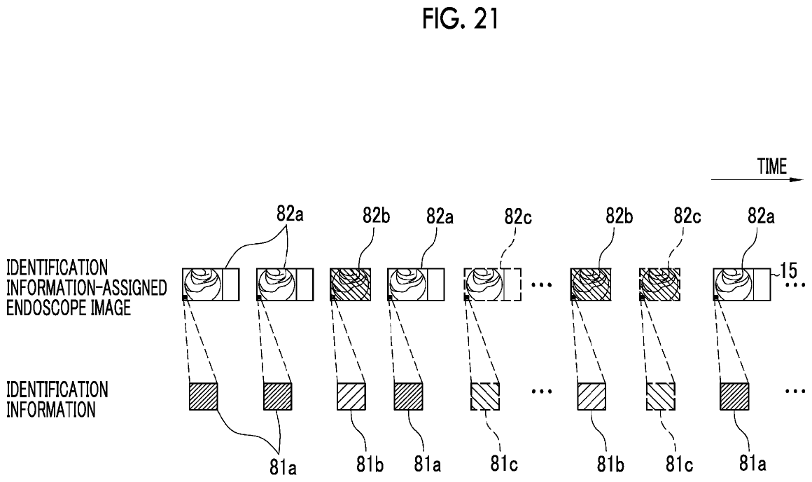

The identification information 81 may be assigned to the complementary frame image 73. As shown in FIG. 21, in this case, identification information 81c indicating the complementary frame image 73 is assigned to the complementary frame image 73, whereby a third identification information-assigned endoscope image 82c is generated. The identification information 81c can be made different from the identification information indicating the type of the endoscope image. As a result, in the medical image processing device 17, it is possible to easily grasp that the image is the complementary frame image 73 through the image data.

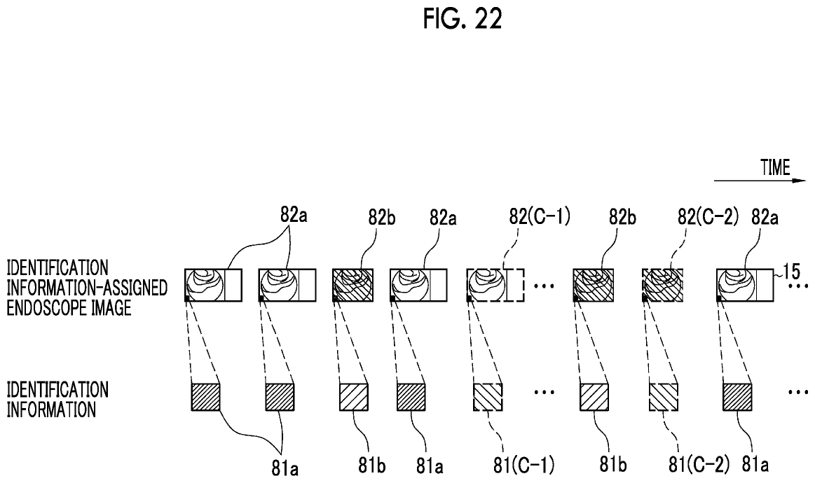

Further, the identification information 81c can include information related to the type of an endoscope image of a duplication source. As shown in FIG. 22, a third identification information-assigned endoscope image 82 (C-1) in which identification information 81 (C-1) is assigned may be generated in the case of the complementary frame image 73 obtained by duplicating the normal image 71, and a third identification information-assigned endoscope image 82 (C-2) in which identification information 81 (C-2) is assigned may be generated in the case of the complementary frame image 73 obtained by duplicating the first image 72. In this case, in the medical image processing device 17, it is possible to easily grasp through the image data what the duplication source of the complementary frame image 73 is, in addition to the fact that it is the complementary frame image 73.

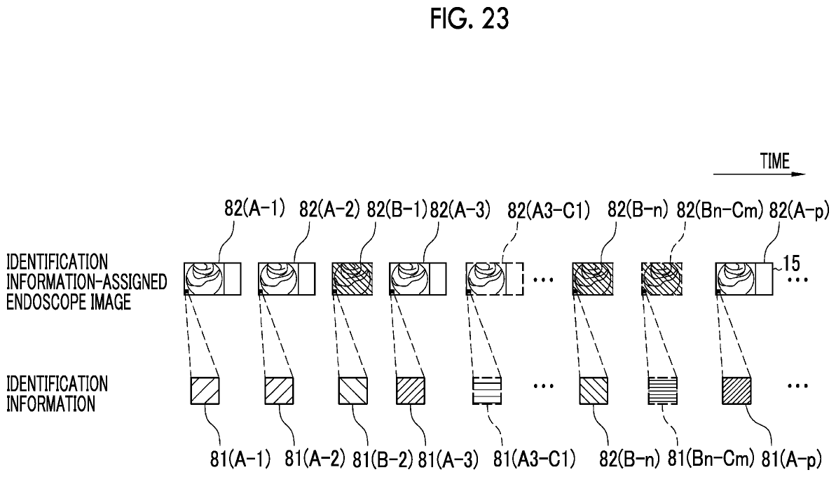

Further, the identification information 81c can include information regarding the imaging order in addition to information regarding the type of the endoscope image of the duplication source. As shown in FIG. 23, a first identification information-assigned endoscope image 82 (A-3) is generated based on an image that is the normal image 71 and that is captured third, and a third identification information-assigned endoscope image 82 (A3-C1) in which identification information 81 (A3-C1) is assigned to the complementary frame image 73 obtained by duplicating the normal image 71 is generated. Similarly, a second identification information-assigned endoscope image 82 (B-n) is generated based on an image that is the first image 72 and that is captured N-th, and a third identification information-assigned endoscope image 82 (Bn-Cm) in which identification information 81 (Bn-Cm) is assigned to the complementary frame image 73 that is obtained by duplicating the first image 72 and that is captured m-th is generated. As a result, in the medical image processing device 17, it is possible to easily grasp through the image data what the duplication source of the complementary frame image 73 is and the imaging order, in addition to the fact that it is the complementary frame image 73.

In the medical image processing device 17, since the complementary frame image 73 can be identified, the complementary frame image 73 can be one of the types of endoscope images. Therefore, the medical image processing device 17 can perform an image processing method corresponding to the complementary frame image 73. Examples of the image processing method for the complementary frame image 73 include a method of performing the same type of image processing as in the original endoscope image of the complementary frame image 73 or a method of not performing image processing on the complementary frame image 73.

Further, for example, in a case where the frame rate is set to a high frame rate by the adjustment of the frame rate conversion section 62, the first identification information-assigned endoscope image 82a, the second identification information-assigned endoscope image 82b, or the third identification information-assigned endoscope image 82c, which is the complementary frame image 73, may be identified, and in a case where the third identification information-assigned endoscope image 82c accounts for a certain percentage or more, the image processing speed for the image processing on the first identification information-assigned endoscope image 82a or the second identification information-assigned endoscope image 82b may be adjusted from the viewpoint of the image processing speed or the like. As described above, by assigning the identification information 81c also to the complementary frame image 73, in the medical image processing device 17, the information on the frame rate can also be grasped using the information of only the image data without acquiring the information on the frame rate, and can be utilized for adjustment of the speed of the image processing, and the like.

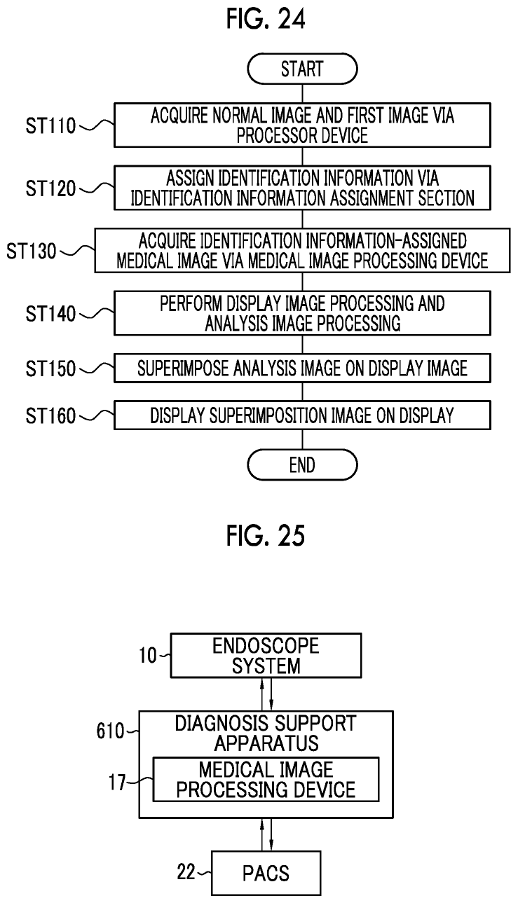

Next, a series of flows of the discrimination of the type of the endoscope image will be described along the flowchart shown in FIG. 24. The observation target is imaged using the endoscope. The normal image 71, which is the display image, and the first image 72, which is the analysis image, are each acquired in a preset frame pattern (step ST110). On the display 15, the normal image 71 is displayed after the frame rate is adjusted. In the processor device 14, the identification information assignment section 61 assigns the identification information 81 to each of the normal image 71 and the first image 72 (step ST120).

The first identification information-assigned endoscope image 82a and the second identification information-assigned endoscope image 82b, both of which have the identification information 81 assigned thereto, are acquired by the identification information-assigned medical image acquisition unit 91 of the medical image processing device 17 (step ST130). The display image processing section 95 performs the display image processing on the first identification information-assigned endoscope image 82a, which is the display image. The image analysis section 96 performs image analysis for obtaining the diagnosis support information on the second identification information-assigned endoscope image 82b, which is the analysis image, by using the machine learning-based analysis model (step ST140). The analysis result creation section 97 generates the analysis result image 101, which shows the result of the image analysis through display. The image superimposition section 98 generates the superimposition image 102 by superimposing the analysis result image 101 on the normal image 71 that is the first identification information-assigned endoscope image 82a subjected to the display image processing (step ST150). The superimposition image 102 is displayed on the display 15 (step ST160).

In the above-described embodiment, the present invention is applied to a case of processing the endoscope image, but the present invention can also be applied to a processor device, a medical image processing device, a medical image processing system, or the like that processes a medical image other than the endoscope image.

In addition, some or all of the image processing unit 56 and/or the central control unit 59 of the endoscope system 10 can be provided in, for example, a diagnosis support apparatus 610 that collaborates with the endoscope system 10 by communicating with the processor device 14. Similarly, some or all of the medical image processing device 17 in the endoscope system 10 can be provided in, for example, the diagnosis support apparatus 610 that collaborates with the endoscope system 10 by communicating with the medical image processing device 17.

Further, some or all of the image processing unit 56 and/or the central control unit 59 in the endoscope system 10 can be provided in, for example, the diagnosis support apparatus 610 that acquires an image captured by the endoscope 12 directly from the endoscope system 10 or indirectly from a picture archiving and communication systems (PACS) 22. Similarly, some or all of the medical image processing device 17 in the endoscope system 10 can be provided in, for example, the diagnosis support apparatus 610 that acquires an image captured by the endoscope 12 directly from the endoscope system 10 or indirectly from the picture archiving and communication systems (PACS) 22, as shown in FIG. 25.

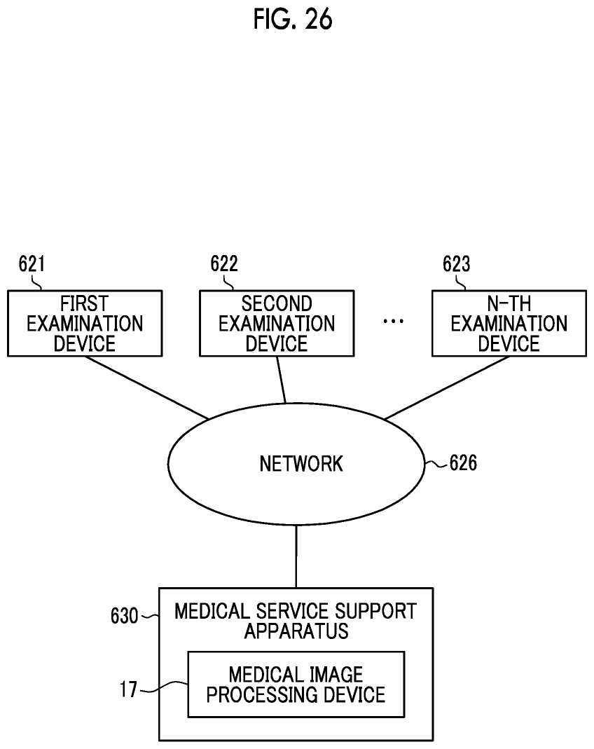

In addition, a medical service support apparatus 630 connected to various examination devices including the endoscope system 10, such as a first examination device 621, a second examination device 622, . . . , and an N-th examination device 623, via a network 626 can be provided with some or all of the image processing unit 56 and/or the central control unit 59, or some or all of the medical image processing device 17 in the endoscope system 10, as shown in FIG. 26.

In the above-described embodiment, the hardware structures of the light source processor, the image processors, which are the first processor and the second processor, and the processing units that execute various types of processing, such as the central control unit 59, the image acquisition unit 51, the DSP 52, the noise reduction unit 53, the memory 54, the signal processing unit 55, the image processing unit 56, the display control unit 57, and the video signal generation unit 58 provided in the processor device 14, and the identification information-assigned medical image acquisition unit 91, the identification information-assigned medical image recognition unit 92, the identification information-assigned medical image processing unit 93, and the display control unit 94 provided in the medical image processing device, are various processors as follows. The various processors include a central processing unit (CPU) that is a general-purpose processor functioning as various processing units by executing software (programs), a programmable logic device (PLD) that is a processor of which a circuit configuration can be changed after manufacturing, such as a field programmable gate array (FPGA), a dedicated electric circuit that is a processor having a circuit configuration exclusively designed to execute various types of processing, and the like.

One processing unit may be composed of one of these various processors or may be composed of a combination of two or more processors of the same type or different types (for example, a plurality of FPGAs or a combination of a CPU and an FPGA). Alternatively, a plurality of processing units may be composed of one processor. A first example in which a plurality of processing units are composed of one processor includes an aspect in which one or more CPUs and software are combined to constitute one processor and the processor functions as a plurality of processing units, as represented by a computer, such as a client or a server. A second example of the configuration includes an aspect in which a processor that realizes all the functions of a system including a plurality of processing units with one integrated circuit (IC) chip is used, as represented by a system on chip (SoC). As described above, various processing units are composed of one or more of the above-described various processors, as the hardware structure.

Furthermore, as the hardware structures of the various processors, more specifically, electric circuitry obtained by combining circuit elements, such as semiconductor elements, may be used.

### EXPLANATION OF REFERENCES

10: endoscope system
12: endoscope
12a: insertion part
12b: operation part
12c: bendable portion
12d: distal end portion
12e: angle knob
12f: zoom operation portion
12g: mode changeover switch
13: light source device
14: processor device
15 display
16: keyboard
17: medical image processing device
18: medical image processing system
20 light source unit
20a V-LED
20b B-LED
20c G-LED
20d R-LED
21: light source processor
22: PACS
30a illumination optical system
30b imaging optical system
41: light guide
42: illumination lens
43: objective lens
44: zoom lens
45 imaging sensor
46: CDS/AGC circuit
47: A/D converter
51: image acquisition unit
52: DSP
53: noise reduction unit
54: memory
55 signal processing unit
56: image processing unit
57, 94: display control unit
58: video signal generation unit
59: central control unit
61: identification information assignment section
62, 99: frame rate conversion section
71: normal image
72: first image
73: complementary frame image
81: identification information
81a: first identification information
81b: second identification information
82: identification information-assigned endoscope image
82a: first identification information-assigned endoscope image
82b: second identification information-assigned endoscope image
83: endoscope image
83a: observation target portion
83b: mask portion
91: identification information-assigned medical image acquisition unit
92: identification information-assigned medical image recognition unit
93: identification information-assigned medical image processing unit
95 display image processing section
96: image analysis section
97: analysis result creation section
98: image superimposition section
101: analysis result image
102: superimposition image
201: main screen
202: first sub screen
203: second sub screen
204: patient information display screen
205: analysis result text display screen
206: past image
610: diagnosis support apparatus
621: first examination device
622: second examination device
623: N-th examination device
626: network
630: medical service support apparatus
ST110 to ST160: step

What is claimed is:

1. A processor device comprising:
a first processor configured to:
acquire a plurality of types of medical images with different imaging conditions; and
generate an identification information-assigned medical image in which a part of data constituting the medical image is assigned as identification information indicating a type of the medical image by changing the part of the data constituting the medical image, or by changing the part of the data constituting the medical image in at least one type of the medical image and not changing the part of the data constituting the medical image in another type of the medical image, according to the type of the medical image,
wherein the data constituting the medical image is data constituting a preset region of the medical image, and
the preset region of the medical image is a mask portion where an observation target does not appears, or an edge portion of a region where the observation target appears, in the medical image.

2. The processor device according to claim 1,
wherein the data constituting the medical image is a pixel value.

3. The processor device according to claim 1,
wherein the plurality of types of medical images include a display image for display on a display and an analysis image for analysis related to diagnostic information.

4. The processor device according to claim 3,
wherein the first processor is configured to assign the identification information to the analysis image by changing a part of data constituting the analysis image, and to assign the identification information to the display image without changing a portion of data constituting the display image, the portion corresponding to the data assigned as the identification information in the analysis image.

5. The processor device according to claim 3,
wherein the first processor is configured to assign the identification information to the display image by changing a part of data constituting the display image, and to assign the identification information to the analysis image without changing a portion of data constituting the analysis image, the portion corresponding to the data assigned as the identification information in the display image.

6. The processor device according to claim 1,
wherein the imaging condition is a spectrum of illumination light.

7. A medical image processing device comprising:
a second processor configured to:
acquire a plurality of types of identification information-assigned medical images in which a part of data constituting a medical image is assigned as identification information;
recognize the type of the identification information-assigned medical image based on the identification information; and
perform control to display the identification information-assigned medical image on a display based on the type of the identification information-assigned medical image,
wherein the identification information is data constituting a preset region of the medical image, and
the preset region of the medical image is a mask portion where an observation target does not appears, or an edge portion of a region where the observation target appears, in the medical image.

8. The medical image processing device according to claim 7,
wherein the plurality of types of identification information-assigned medical images include a display image for display on the display and an analysis image for analysis related to diagnostic information.

9. The medical image processing device according to claim 8,
wherein the second processor is configured to display the display image on a main screen of the display, and to decide whether or not to display the analysis image on a sub screen of the display based on the type of the identification information-assigned medical image and display, on the sub screen of the display, the identification information-assigned medical image that is decided to be displayed.

10. The medical image processing device according to claim 7,
wherein the second processor is configured to perform image processing set for each type of the identification information-assigned medical image on the identification information-assigned medical image based on the type of the identification information-assigned medical image.

11. The medical image processing device according to claim 8,
wherein the second processor is configured to, in a case where the identification information-assigned medical image is the display image, perform display image processing on the display image, and to, in a case where the identification information-assigned medical image is the analysis image, perform analysis image processing on the analysis image.

12. The medical image processing device according to claim 11,
wherein the second processor is configured to perform the analysis image processing using a machine learning-based analysis model.

13. The medical image processing device according to claim 11,
wherein the second processor is configured to create an analysis result image indicating a result of the analysis image processing, and to generate a superimposition image by superimposing the analysis result image on the display image.

14. A medical image processing system comprising:
the processor device according to claim 1; and
the medical image processing device according to claim 7,
wherein the second processor is configured to acquire the plurality of types of identification information-assigned medical images generated by the first processor.

15. A medical image processing system comprising:
the processor device according to claim 1; and
the medical image processing device according to claim 13,
wherein the processor device is configured to acquire the analysis result image indicating the result of the analysis image processing and created by the second processor.

16. The medical image processing system according to claim 15,
wherein the processor device is configured to superimpose the analysis result image on the display image.

17. The medical image processing system according to claim 14,
wherein the processor device is configured to adjust a frame rate of the identification information-assigned medical image, and
the medical image processing device is configured to acquire the identification information-assigned medical image of which the frame rate is adjusted.

18. The medical image processing system according to claim 14,
wherein the processor device or the medical image processing device is configured to adjust a frame rate of an image for display on a display.

19. An endoscope system comprising:
a plurality of light sources that emit light rays having wavelength ranges different from each other;
an endoscope that images a subject illuminated with illumination light emitted from the plurality of light sources; and
the medical image processing system according to claim 14,
wherein the processor device includes a light source processor that is configured to perform control to emit each of a plurality of types of the illumination light having different combinations of light intensity ratios between the plurality of light sources from each other.
